(19) United States
(12) Patent Application Publication
(10) Pub. No.: US 2004/0167226 A1
(43) Pub. Date: Aug. 26, 2004

(54) Methods for the treatment of pain and traumatic injury using benzamides and compositions containing the same

(76) Inventors: Tito A. Serafini, San Mateo, CA (US); Corey S. Goodman, Oakland, CA (US)

Correspondence Address: KLAUBER & JACKSON, 411 HACKENSACK AVENUE, HACKENSACK, NJ, 07601

(21) Appl. No.: 10/736,460

(22) Filed: Dec. 15, 2003

Related U.S. Application Data
(60) Provisional application No. 60/434,022, filed on Dec. 16, 2002.

(51) Int. Cl. A61K 31/165

(52) U.S. Cl. 514/619

(57) ABSTRACT

Methods are disclosed for treating and preventing pain such as neuropathic pain, and traumatic injuries such as traumatic brain injury and acute spinal cord injury, which comprise administering effective amounts of a benzamide compound of formula I:

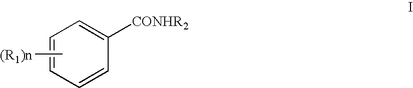

where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5 atoms. Pharmaceutical compositions, dosage forms and methods of administration are set forth.

### CROSS-REFERENCE TO RELATED APPLICATIONS

The present application claims the priority of co-pending provisional U.S. Serial No. 60/434,022, filed on Dec. 16, 2002, the disclosure of which is incorporated by reference herein in its entirety. Applicants claim the benefits of this application under 35 U.S.C. §119(e).

### FIELD OF THE INVENTION

This invention relates to the treatment of conditions such as pain and acute conditions such as traumatic brain injury (TBI) and acute spinal cord injury (SCI). More specifically, it relates to methods and pharmaceutical compositions for the treatment and prophylaxis of pain, TBI and acute spinal cord injury in mammals, including humans.

### BACKGROUND OF THE INVENTION

Neuropathic pain (NP) is a category of chronic pain that has been widely studied. Neuropathic pain occurs when the peripheral and/or central nervous systems are sensitized following an injury to the peripheral system. This initial injury can occur from a wide variety of causes including traumatic physical injury, as well as systemic diseases such as diabetes, herpes zoster, AIDS/HIV, syphilis and various other autoimmune diseases.

Examples of pain syndromes of this class include post herpetic neuralgia, neuritis, temporomandibular disorder, myofacial pain, back pain, and pain induced by inflammatory conditions. Neuropathic pain may occur in all body regions. For example, it may originate from the dental region. It may originate in regions which have suffered burn injury which often leads to neuropathic hyperalgesia in the affected body area. Neuropathic pain can also arise from neuralgia which in its acute phase involves intraneural inflammation which can cause damage to primary afferent axons, this inducing neuropathic pain. Neuropathic pain may also be induced by diabetic conditions (diabetic neuropathy). Neuropathy of primary afferent axons in long nerves is found in diabetic patients. Nociceptor sensitization may ensue.

Accordingly, a need exists for novel classes of therapeutic compounds which effectively treat the pain associated with joint disease such as arthritis, and a need exists to treat neuropathic pain which may present in the presence and absence of arthritis, as well as conditions such as traumatic brain injury and acute spinal cord injury.

### SUMMARY OF THE INVENTION

It has now been found that certain benzamide compounds have activity in the treatment and prophylaxis of pain and traumatic injury.

Accordingly, a first aspect of the invention provides for a pharmaceutical composition for the treatment of pain or traumatic injury in a mammal in need of such therapy, comprising an effective pain-treating amount or traumatic injury-treating amount of a benzamide compound in a pharmaceutically acceptable carrier. One aspect of this invention provides a pharmaceutical composition for the treatment of and prophylaxis of both conditions. These materials and their use in the treatment of Parkinson's disease and related neurological conditions are described in International Application No. PCT/US95/04278.

In a preferred embodiment, the benzamide is a material of Formula I:

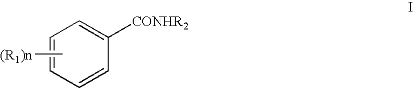

where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5.

In another preferred embodiment, the benzamide is selected from the group consisting of acetamidobenzamides, aminobenzamides and nitrobenzamides.

In yet another preferred embodiment, the benzamide is selected from the group consisting of:

N-tert-butyl-4-acetamidobenzamide (Compound A);

N-iso-propyl-4-acetamidobenzamide (Compound B);

N-tert-amyl-4-acetamidobenzamide (Compound C);

N-cyclopropylmethyl-4-acetamidobenzamide (Compound E);

N-iso-propyl-4-nitrobenzamide (Compound F);

N-tert-butyl-3-nitrobenzamide (Compound G);

N-tert-butyl-2-nitrobenzamide (Compound H);

N-n-butyl-4-nitrobenzamide (Compound J);

N-n-propyl-4-nitrobenzamide (Compound K);

N-tert-butyl-3,5-dinitrobenzamide (Compound L);

N-1-methylpropyl-4-nitrobenzamide (Compound M);

N-tert-butyl-4-aminobenzamide (Compound N);

N-tert-butyl-3-aminobenzamide (Compound, P); and

N-tert-butyl 4-nitrobenzamide (Compound Q).

In another preferred embodiment, the benzamide is N-tert-butyl-4-acetamidobenzamide (Compound A).

In a further preferred embodiment, the pharmaceutical compositions may be delivered/administered via a route selected from the group consisting of topical, oral, caudal, subcutaneous, intramuscular, intravenous, perineural, intraperitoneal, epidural intranasal, intracranial, local intracerebral, intrathecal, intraventricular, transdermal, and combinations thereof. In a yet further preferred embodiment, the compositions may be delivered directly to the central nervous system of the subject, without adversely affecting other tissues or organ systems, for example via intracranial, local intracerebral, intrathecal, and intraventricular delivery/administration. In yet another preferred embodiment, the pharmaceutical composition is administered by an infusion pump or by an aerosol spray.

In another preferred embodiment, the pharmaceutical composition is administered over a period of at least several days. In yet another preferred embodiment, the pharmaceutical composition is administered over a period of at least four weeks. In another preferred embodiment, the pharmaceutical composition is administered over a period of at least three months. In yet another preferred embodiment, the pharmaceutical composition is administered on an as needed basis.

In another preferred embodiment, the pharmaceutical composition is administered as a sustained release formulation or in a formulation that is capable of crossing the blood brain barrier.

In another preferred embodiment, the patient/subject to be treated is a mammal, preferably a human, although use of the compounds of the present invention and pharmaceutical compositions comprising the benzamides for treatment of such conditions in other mammals is also conceived. Included in this are domestic animals such as cats and dogs, as well as non-domestic animals.

A second aspect of the present invention provides for a method of treating traumatic injury comprising administering to a patient an effective amount of a pharmaceutical composition comprising a benzamide compound of the formula I:

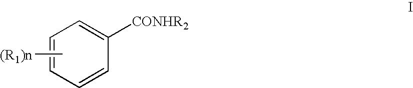

where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5 atoms in a pharmaceutically acceptable carrier.

In another preferred embodiment, the benzamide may be selected from the group consisting of:

N-tert-butyl-4-acetamidobenzamide (Compound A);

N-iso-propyl-4-acetamidobenzamide (Compound B);

N-tert-amyl-4-acetamidobenzamide (Compound C);

N-methylcyclopropyl-4-acetamidobenzamide (Compound E);

N-iso-propyl-4-nitrobenzamide (Compound F);

N-tert-butyl-3-nitrobenzamide (Compound G);

N-tert-butyl-2-nitrobenzamide (Compound H);

N-n-butyl-4-nitrobenzamide (Compound J);

N-n-propyl-4-nitrobenzamide (Compound K);

N-tert-butyl-3,5-dinitrobenzamide (Compound L);

N-1-methylpropyl-4-nitrobenzamide (Compound M);

N-tert-butyl-4-aminobenzamide (Compound N);

N-tert-butyl-3-aminobenzamide (Compound P); and

N-tert-butyl 4-nitrobenzamide (Compound Q).

In another preferred embodiment, R₁ is amino, acylamino (NHCOR₃), or nitro.

In another preferred embodiment, R₂ is t-Bu.

In another preferred embodiment, n is 1.

In another preferred embodiment, n is 1, R₁ is 4-amino, 4-acylamino (NHCOR₃), or 4-nitro and R₂ is t-Bu.

In a yet further preferred embodiment, the benzamide is N-tert-butyl-4-acetamidobenzamide (Compound A).

In another preferred embodiment, the traumatic injury is an injury to the central nervous system. The traumatic injury may be selected from the group consisting of traumatic brain injury and spinal cord injury. The spinal cord injury may be an acute or chronic spinal cord injury. The preferred patient may be a human.

In another preferred embodiment, the composition for treating traumatic injury may be delivered/administered orally or parenterally. In yet another preferred embodiment, the parenteral administration may be selected from the group consisting of topical, caudal, subcutaneous, intramuscular, intravenous, perineural, intraperitoneal, epidural, intranasal, intracranial, local intracerebral, intrathecal, intraventricular, transdermal, and combinations thereof. In yet another preferred embodiment, the administration is by infusion pump or by rectal delivery.

A third aspect of the invention provides for a method for treating pain in a mammalian subject in need thereof, comprising delivering/administering to said subject an effective pain-treating amount of a pharmaceutical composition comprising a benzamide.

In a preferred embodiment, the benzamide has the formula set forth in 1:

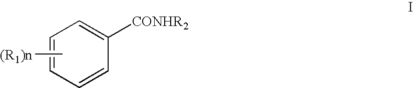

where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5 atoms in a pharmaceutically acceptable carrier.

In another preferred embodiment, the benzamide may be selected from the group consisting of:

N-tert-butyl-4-acetamidobenzamide (Compound A);

N-iso-propyl-4-acetamidobenzamide (Compound B);

N-tert-amyl-4-acetamidobenzamide (Compound C);

N-methylcyclopropyl-4-acetamidobenzamide (Compound E);

N-iso-propyl-4-nitrobenzamide (Compound F);

N-tert-butyl-3-nitrobenzamide (Compound G);

N-tert-butyl-2-nitrobenzamide (Compound H);

N-n-butyl-4-nitrobenzamide (Compound J);

N-n-propyl-4-nitrobenzamide (Compound K);

N-tert-butyl-3,5-dinitrobenzamide (Compound L);

N-1-methylpropyl-4-nitrobenzamide (Compound M);

N-tert-butyl-4-aminobenzamide (Compound N);

N-tert-butyl-3-aminobenzamide (Compound P); and

N-tert-butyl 4-nitrobenzamide (Compound Q).

In another preferred embodiment, R₁ is amino, acylamino (NHCOR₃), or nitro.

In another preferred embodiment, R₂ is t-Bu.

In another preferred embodiment, n is 1.

In another preferred embodiment, n is 1, R₁ is 4-amino, 4-acylamino (NHCOR₃), or 4-nitro and R₂ is t-Bu.

In a yet further preferred embodiment, the benzamide is N-tert-butyl-4-acetamidobenzamide (Compound A).

In another preferred embodiment, the pain is acute pain. The acute pain may be selected from the group consisting of post-operative pain, shock, pain resulting from inflammation, pain resulting from trauma, acute breakthrough pain associated with a chronic pain condition, and combinations thereof.

In another preferred embodiment, the pain is chronic pain. The chronic pain may be selected from the group consisting of headache pain, back pain, sciatica, cancer pain, arthritis pain, neuropathic pain, psychogenic pain, and combinations thereof.

In another preferred embodiment, the pain is associated with a condition selected from the group consisting of diabetic neuropathy, herpes zoster, fibromyalgia A/DS, syphilis, neuritis, temporomandibular disorder, back pain, myofacial pain, acute spinal cord injury, traumatic brain injury, cancer pain, visceral pain, intraneural inflammation, optic neuropathy, neuropathy secondary to nerve trauma, post herpetic neuralgia, reflex sympathetic dystrophy, and ankylosing spondylitis.

The pharmaceutical compositions may be administered over a period of at least several days or over a period of at least four weeks, or for several months. The length of time of treatment would be dependent upon the nature of the injury or pain condition (pain inducing pathology) and would depend on whether the injury or pain condition (pain inducing pathology) was acute, sub-chronic or chronic. For example, the acute stage can extend from days to weeks, the sub-chronic stage can extend eg. from six months to a year and the chronic stage would account for recurring episodes beyond one year forward, which is tantamount to a permanent condition. For treatment of neuropathic pain, the compounds and compositions of the present invention may be administered for several days to several weeks; for treatment of spinal cord injury, the treatment may be initiated immediately after injury and be administered for several weeks thereafter. Preferably, treatment of the spinal cord injury during the acute stage, ie. shortly after injury, would commence immediately after the injury occurs, or at least within the first few hours to days. For treatment of the pain associated with chronic spinal cord injuries, the compounds and compositions of the present invention may be administered on an as needed basis, ie. for several days, or weeks or months after the injury. In addition, the compounds and compositions of the present invention may be used to treat the neuropathic pain associated with chemotherapy and as such, treatment would be given on an as needed basis. In addition, the compounds and compositions of the present invention may be given prior to receiving chemotherapy, that is, as a prophylactic therapy for prevention of the pain and the neuropathy that follows chemotherapy.

A fourth aspect of the invention provides for a method for producing local anesthesia or analgesia in a mammal, the method comprising administering to a mammal a therapeutically effective amount of a pharmaceutical composition comprising a benzamide.

In a preferred embodiment, the benzamide has the formula set forth in I:

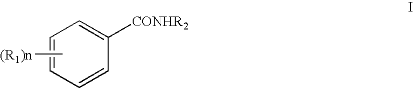

where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5 atoms in a pharmaceutically acceptable carrier.

In another preferred embodiment, the benzamide may be selected from the group consisting of:

N-tert-butyl-4-acetamidobenzamide (Compound A);

N-iso-propyl-4-acetamidobenzamide (Compound B);

N-tert-amyl-4-acetamidobenzamide (Compound C);

N-methylcyclopropyl-4-acetamidobenzamide (Compound E);

N-iso-propyl-4-nitrobenzamide (Compound F);

N-tert-butyl-3-nitrobenzamide (Compound G);

N-tert-butyl-2-nitrobenzamide (Compound H);

N-n-butyl-4-nitrobenzamide (Compound J);

N-n-propyl-4-nitrobenzamide (Compound K);

N-tert-butyl-3,5-dinitrobenzamide (Compound L);

N-1-methylpropyl-4-nitrobenzamide (Compound M);

N-tert-butyl-4-aminobenzamide (Compound N);

N-tert-butyl-3-aminobenzamide (Compound P); and

N-tert-butyl 4-nitrobenzamide (Compound Q).

In another preferred embodiment, R₁ is amino, acylamino (NHCOR₃), or nitro.

In another preferred embodiment, R₂ is t-Bu.

In another preferred embodiment, n is 1.

In another preferred embodiment, n is 1, R₁ is 4-amino, 4-acylamino (NHCOR₃), or 4-nitro and R₂ is t-Bu.

In a yet further preferred embodiment, the benzamide is N-tert-butyl-4-acetamidobenzamide (Compound A).

In a yet further preferred embodiment, the local anesthesia or analgesia is effective for treatment of conditions associated with increased sensory functions, said conditions selected from the group consisting of hyperesthesia, hyperalgesia, dyesthesia, allodynia, tinnitus and ganglionic dysfunction.

In a yet further preferred embodiment, the local anesthesia or analgesia is administered topically to a skin region having or manifesting neuropathic pain. The treatment of neuropathic pain in a skin region may be accomplished by the application of a transdermal patch containing the pharmaceutical compositions of the present invention.

A fifth aspect of the invention provides for a method for the prophylactic treatment of patients susceptible to outbreaks of shingles or for patients scheduled to receive chemotherapy comprising administering a therapeutically effective amount of a composition containing a benzamide.

In a preferred embodiment, the benzamide has the formula set forth in I:

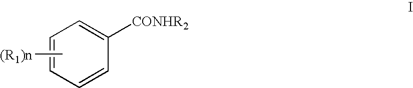

where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5 atoms in a pharmaceutically acceptable carrier.

In another preferred embodiment, the benzamide may be selected from the group consisting of:

N-tert-butyl-4-acetamidobenzamide (Compound A);

N-iso-propyl-4-acetamidobenzamide (Compound B);

N-tert-amyl-4-acetamidobenzamide (Compound C);

N-methylcyclopropyl-4-acetamidobenzamide (Compound E);

N-iso-propyl-4-nitrobenzamide (Compound F);

N-tert-butyl-3-nitrobenzamide (Compound G);

N-tert-butyl-2-nitrobenzamide (Compound H);

N-n-butyl-4-nitrobenzamide (Compound J);

N-n-propyl-4-nitrobenzamide (Compound K);

N-tert-butyl-3,5-dinitrobenzamide (Compound L);

N-1-methylpropyl-4-nitrobenzamide (Compound M);

N-tert-butyl-4-aminobenzamide (Compound N);

N-tert-butyl-3-aminobenzamide (Compound P); and

N-tert-butyl 4-nitrobenzamide (Compound Q).

In another preferred embodiment, R₁ is amino, acylamino (NHCOR₃), or nitro.

In another preferred embodiment, R₂ is t-Bu.

In another preferred embodiment, n is 1.

In another preferred embodiment, n is 1, R₁ is 4-amino, 4-acylamino (NHCOR₃), or 4-nitro and R₂ is t-Bu.

In a yet further preferred embodiment, the benzamide is N-tert-butyl-4-acetamidobenzamide (Compound A).

Other aspects of the invention, and associated objects and advantages will become apparent from a review of the ensuing description taken in conjunction with the following illustrative drawings.

### BRIEF DESCRIPTION OF THE DRAWINGS

FIG. 1: A graph showing plasma concentration of Compound N as a function of time in rats given 30 mg/kg of Compound N orally.

FIG. 2: A graph showing that Compound A has activity preventing Taxol-induced pain in a mouse model.

FIG. 3: A graph showing the analgesic effects of Compound A on Taxol-induced mechanical allodynia. Open box: mice received 100 mL per 20 grams (body weight) of 75% PEG-200 as vehicle control via i.p administration, N=9; hatched box: mice received Compound A at 50 mg/kg dose via i.p. administration, N=8. The inhibition of mechanical allodynia was measured at 1, 6, 24, and 48 hour post compound dosing. The percentage of paw withdraw response was accessed using von Frey filament 0.6 gram (von Frey Number 3.84). The error bar represents +/−SEM.

FIG. 4: A graph showing the results of a dose response study to test the analgesic effect of Compound A in the Taxol neuropathic pain mouse model. Vehicle control group, N=9, 100 uL of 9% Cremophor EL/1% ETOH/90% saline per 20 mg of body weight of mouse was administered via i.p.; 5 mg/kg dose of Compound A: N=10; 10 mg/kg dose of Compound A: N=10; and 20 mg/kg dose: N=10. Drug at different dose was administered to animal via i.p. The percentage of response was measured using von Frey filament with 0.6 gram of force (von Frey Number=3.84) at 1, 3, 6, and 24 hour postdosing timepoints. Baseline was obtained before the animal exposed to taxol and the paw withdraw response 21 days post taxol treat ment was measured. Error bar represents +/−SEM.

FIG. 5: A graph showing the results of chronic dosing of Compound A in the Taxol-induced neuropathic pain mouse model. 12 animals in each group: vehicle control, 5 mg/kg/day, and 10 mg/kg/day, received either vehicle or drug at different dose. The drug or vehicle was administered as a single dose per day in the morning via i.p. injection for 12 consecutive days. The percentage of paw withdraw were accessed using von Frey filament (0.6 gram) prior to drug injection (pre) and 3-hour post drug injection (post) on each day during the testing period, with exception of day 7 and day 7, which were weekends. Taxol treatment was continued throughout the course in the late afternoon. Error bar represents +/−SEM.

FIG. 6: Dose response of Compound A on Taxol induced neuropathic pain in a rat model. After 15 days of Taxol treatment, rats were randomly assigned to 4 groups. Vehicle control group, N=6, received 1 ml/kg of 1% methycellulose; 6 rats received Compound A at 20 mg/kg dose, 7 rats received 50 mg/kg; and 7 rats received 50/kg of Gabapentine as positive control. The vehicle or drug was administered via oral gavage feeding (p.o). The paw withdraw threshold was obtained at 1, 2, 3, 6, and 24 hours postdosing by up-down measurement using a set of von Frey Filaments. Error bar stands for +/−SEM.

FIG. 7: Results of Cognition Tests from study number 1 using Compound A at 20 mg/kg/BID in the fluid percussion injury model in rats. SV=Sham vehicle; SD=Sham drug; IV=Injured vehicle; ID=Injured drug.

FIG. 8: Results of Composite Neuroscore from study number 1 using Compound A at 20 mg/kg/BID in the fluid percussion injury model in rats. SV=Sham vehicle; SD=Sham drug; IV=Injured vehicle; ID=Injured drug.

FIG. 9: Results of the beam balance test from study number 1 using Compound A at 20 mg/kg/BID in the fluid percussion injury model in rats. SV=Sham vehicle; SD=Sham drug; IV=Injured vehicle; ID=Injured drug.

FIG. 10: Results of the rotating pole test from study number 1 using Compound A at 20 mg/kg/BID in the fluid percussion injury model in rats. SV=Sham vehicle; SD=Sham drug; IV=Injured vehicle; ID=Injured drug.

FIG. 11: Results of Cognition Test results from study number 2 using Compound A at 10 and 20 mg/kg/BID in the fluid percussion injury model in rats. IV=Injured vehicle; SV=Sham vehicle.

FIG. 12: Results of Composite Neuroscore from study number 2 using Compound A at 10 and 20 mg/kg/BID in the fluid percussion injury model in rats. IV=Injured vehicle; SV=Sham vehicle.

FIG. 13: Results of beam balance test from study number 2 using Compound A at 10 and 20 mg/kg/BID in the fluid percussion injury model in rats. WV=Injured vehicle; SV=Sham vehicle.

FIG. 14: Results of CA3 cell counts and lesion volume in study number 2 using Compound A at 10 and 20 mg/kg/BID in the fluid percussion injury model in rats.

FIG. 15: Results of the T8 Hemisection 14 Day Study; average of BBB Score after Dorsal Hemisection Lesion (1.5 mm in depth from dorsal surface) in rats.

FIG. 16: Results of the T8 Over-hemisection 21 Day Study; average of BBB Score after Over Dorsal Hemisection Lesion (2 mm in depth from dorsal surface which represents a more severe injury than the previous hemisection) in rats.

FIG. 17: Results of the T8 Over-hemisection 5 Week Study; average of BBB Score after Over Dorsal Hemisection Lesion for up to 5 weeks in rats.

FIG. 18: Results of the Contusion Model; average of BBB Score after a contused spinal cord injury in rats.

FIG. 19: Immunohistochemistry of neurofilament on transverse spinal cords sections (2 mm rostral to epicenter of lesion).

FIG. 20: Immunohistochemistry of neurofilament on transverse spinal cords sections (2 mm rostral to epicenter of lesion).

FIG. 21: Immunohistochemistry for inflammatory markers TNFα and iNOS on sections of contused spinal cord (72 h post-injury, 1 mm caudal to epicenter).

### DETAILED DESCRIPTION OF THE INVENTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference in their entirety.

Definitions

When describing the benzamides, pharmaceutical compositions and methods of this invention, the following terms have the following meanings unless otherwise specified. The remaining terms used herein have the meanings recognized and known to those of skill in the art, however, for convenience and completeness, particular terms and their meanings are set forth below.

“Acyl” refers to the group —C(O)R where R is hydrogen, alkyl, alkenyl, aryl, aralkyl or cycloalkyl.

“Alkanoylamido” or “acylamino” refers to the group —NRC(O)R where each R is independently hydrogen, alkyl, alkenyl, aryl, aralkyl or cycloalkyl.

“Alkyl” refers to a monovalent branched or unbranched saturated hydrocarbon group preferably having from 1 to about 10 carbon atoms, more preferably from 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to an alkyl group having from 1 to 6 carbon atoms.

“Amino” refers to the group —NH₂. “Substituted amino” refers to the group —N(R)₂ where up to one R is hydrogen and at least one R is independently selected from the group consisting of alkyl, alkenyl, alkynyl, aryl, cycloalkyl, and where both R groups are joined to form an alkylene group. When one R is non hydrogen this is a “secondary” amino, when both R's are non hydrogen this is a “tertiary” amino.

“Halo” or “halogen” refers to fluoro, chloro, bromo and iodo. Preferred halo groups are either fluoro or chloro.

“Nitro” or “nitrate” refers to the group —NO₂.

“Pharmaceutically-acceptable salt” refers to any salt of a compound of this invention which retains its biological properties and which is not biologically or otherwise undesirable. Such salts may be derived from a variety of organic and inorganic counter-ions well known in the art and include, by way of example illustration, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. The term “pharmaceutically-acceptable cation” refers to a pharmaceutically acceptable cationic counter-ion of an acidic functional group. Such cations are exemplified by sodium, potassium, calcium, magnesium, ammonium, tetraalkylammonium cations, and the like.

“Treatment” or “treating” refers to the administration of medicine or the performance of medical procedures with respect to a patient, for either prophylaxis (prevention) or to cure the infirmity or malady in the instance where the patient is afflicted.

A “therapeutically effective amount” is an amount sufficient to decrease or prevent the symptoms associated with a medical condition or infirmity or to normalize body functions in diseases or disorders that result in impairment of specific bodily functions. As related to the present application, an “effective pain-treating amount” is an amount sufficient to reduce the pain associated with the neurological disorder being treated, or an “effective traumatic injury-treating amount” is an amount sufficient to result in improvement of sensory or motor function in subjects having a spinal cord injury or traumatic brain injury.

“Local administration” means direct administration by a non-systemic route at or in the vicinity of the site of an affliction, disorder, or perceived pain.

“Slow or sustained release formulation” refers to a formulation designed to release a therapeutically effective amount of a drug or other active agent such as a polypeptide or a synthetic compound over an extended period of time, with the result being a reduction in the number of treatments necessary to achieve the desired therapeutic effect. In the matter of the present invention, a slow release formulation would decrease the number of treatments necessary to achieve the desired effect in terms of reduction in pain, or an improvement in motor or sensory function in patients in need of such therapy, for example, in spinal cord injured patients or in patients suffering from traumatic brain injury.

The terms “patient” and “subject” mean all animals including humans. Examples of patients or subjects include humans, cows, dogs, cats, goats, sheep, and pigs.

“Herpes zoster” or “varicella-zoster” is a common infection caused by the human herpesvirus 3, the same virus that causes varicella (i.e., chickenpox). This virus is also responsible for the painful condition known as shingles. Shingles occurs in people who have had chickenpox and represents a reactivation of this dormant herpes virus in neurosensory ganglia. The disease generally affects the elderly, although it occasionally occurs in younger and/or immunodeficient individuals. The first sign is usually a tingling feeling, itchiness, or stabbing pain on the skin. After a few days, a rash appears as a band or patch of raised dots on the side of the trunk or face. The rash develops into small, fluid-filled blisters which begin to dry out and crust over within several days. When the rash is at its peak, symptoms can range from mild itching to extreme and intense pain. Contact with a person with shingles may cause chickenpox (but not shingles) in someone who has never had chickenpox before.

“Fibromyalgia” is a chronic condition characterized by fatigue and widespread pain in the muscles, ligaments and tendons. Previously, the condition was known by other names such as fibrositis, chronic muscle pain syndrome, psychogenic rheumatism and tension myalgias. The pain occurs in areas called “tender points.” Common tender points are the front of the knees, the elbows, the hip joints and around the neck. Increased sensitivity to pain is the main symptom of fibromyalgia. Patients with fibromyalgia may have some degree of constant pain, but the pain may get worse in response to activity, stress, weather changes and other factors. They may also have a deep ache or a burning pain, or may have muscle tightening or spasms. Many people have migratory pain (pain that moves around the body). Patients with fibromyalgia may also experience fatigue, which may be mild or severe. Patients may have feelings of numbness or tingling in parts of their body, or a feeling of poor blood flow in some areas. Many people are very sensitive to odors, bright lights, loud noises and even medicines. Headaches and jaw pain are also common. In addition, patients may have dry eyes or difficulty focusing on nearby objects. Problems with dizziness and balance may also occur. Some people have chest pain, a rapid or irregular heartbeat, or shortness of breath. Digestive symptoms are also common in fibromyalgia and include difficulty swallowing, heartburn, gas, cramping abdominal pain, and alternating diarrhea and constipation. Some people have urinary complaints, including frequent urination, a strong urge to urinate and pain in the bladder area. Women with fibromyalgia often have pelvic symptoms, including pelvic pain, painful menstrual periods and painful sexual intercourse.

“Hyperalgesia” is a condition in which there is an increased response to a stimulus that is normally painful. One form of hyperalgesia is visceral hyperalgesia, extreme sensitivity to something painful in the internal organs, usually those of the belly. Visceral hyperalgesia can also mean an increased awareness of the normal movements of internal organs, such as the intestines. It is a common symptom of people with irritable bowel syndrome, a disorder of the intestines with various signs and symptoms, and no clear biological cause.

“Neuropathic pain” is pain that results from a disturbance of function or pathologic change in a nerve; in one nerve mononeuropathy; in several nerves, mononeuropathy multiplex; if diffuse and bilateral, polyneuropathy. Neuropathic pain is initiated or caused by a primary lesion or dysfunction in the nervous system. Peripheral neuropathic pain occurs when the lesion or dysfunction affects the peripheral nervous system. Central pain may be retained as the term when the lesion or dysfunction affects the central nervous system. Neuropathic pain comes from aberrant signaling in the pain transmission or pain modulation pathways. The quality of the pain is typically burning and often there is a paroxysmal quality such as shooting, jabbing or shock-like pain. Examples of neuropathic pain include: monoradiculopathies, trigeminal neuralgia, postherpetic neuralgia, phantom limb pain, complex regional pain syndromes and the various peripheral neuropathies. Neuropathic pain tends to be only partially responsive to opioid therapy. Neuropathic pain, in contrast to nociceptive pain, is described as “burning”, “electric”, “tingling”, and “shooting” in nature. It can be continuous or paroxysmal in presentation. Whereas nociceptive pain is caused by the stimulation of peripheral of A-delta and C-polymodal pain receptors, by algogenic substances (eg. histamine bradykinin, substance P, etc.) neuropathic pain is produced by damage to, or pathological changes in the peripheral or central nervous systems.

“Allodynia” is pain due to a stimulus which does not normally provoke pain. The term allodynia was originally introduced to separate from hyperalgesia and hyperesthesia, the conditions seen in patients with lesions of the nervous system where touch, light pressure, or moderate cold or warmth evoke pain when applied to apparently normal skin. It is important to recognize that allodynia involves a change in the quality of a sensation, whether tactile, thermal, or of any other sort. The original modality is normally non-painful, but the response is painful. There is thus a loss of specificity of a sensory modality. By contrast, hyperalgesia (q.v.) represents an augmented response in a specific mode, viz., pain. With other cutaneous modalities, hyperesthesia is the term which corresponds to hyperalgesia, and as with hyperalgesia, the quality is not altered. In allodynia the stimulus mode and the response mode differ, unlike the situation with hyperalgesia. This distinction should not be confused by the fact that allodynia and hyperalgesia can be plotted with overlap along the same continuum of physical intensity in certain circumstances, for example, with pressure or temperature.

“Neuropathy” is a disturbance of function or pathological change in a nerve: in one nerve, mononeuropathy; in several nerves, mononeuropathy multiplex; if diffuse and bilateral, polyneuropathy. “Neuritis” is a special case of neuropathy and the term is now reserved for inflammatory processes affecting nerves. Neuropathy is not intended to cover cases like neurapraxia, neurotmesis, section of a nerve, or transitory impact like a blow, stretching, or an epileptic discharge. The term “neurogenic” applies to pain due to such temporary perturbations.

“Optic neuropathy” involves damage or destruction of the optic nerve. It is most often caused by damage to a local area resulting from injury or trauma, although occasionally systemic disorders may cause isolated nerve damage. The usual causes are direct trauma, prolonged pressure on the nerve and compression of the nerve by swelling or injury to nearby structures. The damage includes destruction of the myelin sheath (covering) of the nerve or of part of the nerve cell (the axon). This damage slows or prevents conduction of impulses through the nerve.

“Hyperesthesia” is increased sensitivity to stimulation, excluding the special senses. The stimulus and locus should be specified. Hyperesthesia may refer to various modes of cutaneous sensibility including touch and thermal sensation without pain, as well as to pain. The word is used to indicate both diminished threshold to any stimulus and an increased response to stimuli that are normally recognized. Allodynia is suggested for pain after stimulation which is not normally painful. Hyperesthesia includes both allodynia and hyperalgesia, but the more specific terms should be used wherever they are applicable.

“Acute” pain refers to pain that comes on suddenly and presents with symptoms that appear, change, or worsen rapidly. In acute pain, a strong emphasis is placed on continual assessment of pain intensity using formal rating scales like 0-10, or a visual analog scale where intensity is marked on a 10 cm line from NO PAIN to WORST POSSIBLE PAIN. Such scales and formalized intensity ratings are important in a hospital setting where care is delivered by many, and where doses and drugs are continually changing. In chronic pain management, intensity scores play a lesser role to the role of assessing impairment, function, impact of pain and relative improvement in pain.

“Chronic” pain is pain that persists far beyond the expected recovery time. Chronic pain syndrome is a condition in which chronic pain has substantially interferred with a person's ability to function in normal life roles, and has eroded the pain sufferer's self-esteem, well-being, and relationships. Chronic pain is pain that has been present for about three to six months.

“Acute pain” and “chronic pain” differ in their etiology, pathophysiology, diagnosis and treatment. Acute pain is self-limiting and serves a protective biological function by acting as a warning of on-going tissue damage. It is a symptom of a disease process experienced in or around the injured or diseased tissue. Associated psychological symptoms are minimal and are usually limited to mild anxiety. Acute pain is nociceptive in nature, and occurs secondary to chemical, mechanical and thermal stimulation of A-delta and C-polymodal pain receptors. Chronic pain, on the other hand, serves no protective biological function. Rather than being the symptom of a disease process, chronic pain is itself a disease process. Chronic pain is unrelenting and not self-limiting and as stated earlier, can persist for years and even decades after the initial injury. Chronic pain can be refractory to multiple treatment modalities. If chronic pain is inadequately treated, associated symptoms can include chronic anxiety, fear, depression, sleeplessness and impairment of social interaction. Chronic, non-malignant pain is predominately neuropathic in nature and involves damage either to the peripheral or central nervous systems.

Nociceptive and neuropathic pain are caused by different neuro-physiological processes, and therefore tend to respond to different treatment modalities. Nociceptive pain is mediated by receptors on A-delta and C-fibers which are located in skin, bone, connective tissue, muscle and viscera. These receptors serve a biologically useful role at localizing noxious chemical, thermal and mechanical stimuli. Nociceptive pain can be somatic or visceral in nature. Somatic pain tends to be well localized, constant pain that is described as sharp, aching, throbbing, or gnawing. Examples of nociceptive pain include: post-operative pain, pain associated with trauma, and the chronic pain of arthritis. Nociceptive pain usually responds to opioids and non-steroidal anti-inflammatories (NSAIDS). Examples of pathological changes include prolonged peripheral or central neuronal sensitization, central sensitization related damage to nervous system inhibitory functions, and abnormal interactions between the somatic and sympathetic nervous systems. The hallmarks of neuropathic pain are chronic allodynia and hyperalgesia. Allodynia is defined as pain resulting from a stimulus that ordinarily does not elicit a painful response (eg. light touch). Hyperalgesia is defined as an increased sensitivity to a normally painful stimuli. Primary hyperalgesia, caused by sensitization of C-fibers, occurs immediately within the area of the injury. Secondary hyperalgesia, caused by sensitization of dorsal horn neurons, occurs in the undamaged area surrounding the injury.

“Visceral pain” refers to a pain that tends to be vague in distribution, paroxysmal in nature and is usually described as deep, aching, squeezing and colicky in nature.

“Traumatic brain injury (TBI)” occurs when a sudden physical assault on the head causes damage to the brain. The damage can be focal, confined to one area of the brain, or diffuse, involving more than one area of the brain. TBI can result from a closed head injury or a penetrating head injury. A closed head injury occurs when the head suddenly and violently hits an object, but the object does not break through the skull. A penetrating head injury occurs when an object pierces the skull and enters the brain tissue. Several types of traumatic injuries can affect the head and brain. A skull fracture occurs when the bone of the skull cracks or breaks. A depressed skull fracture occurs when pieces of the broken skull press into the tissue of the brain. This can cause bruising of the brain tissue, called a contusion. A contusion can also occur in response to shaking of the brain within the confines of the skull, an injury called “countrecoup.” Shaken baby syndrome is a severe form of head injury that occurs when a baby is shaken forcibly enough to cause extreme countrecoup injury. Damage to a major blood vessel within the head can cause a hematoma, or heavy bleeding into or around the brain. The severity of a TBI can range from a mild concussion to the extremes of coma or even death. A coma is a profound or deep state of unconsciousness. Symptoms of a TBI may include headache, nausea, confusion or other cognitive problems, a change in personality, depression, irritability, and other emotional and behavioral problems. Some people may have seizures as a result of a TBI.

“Spinal cord injury” refers to any injury to the spinal cord via blunt or penetrating trauma. Extreme flexion or extension (particularly in the neck) of the spine can result in traction on the spinal cord with subsequent injury and the development of neurologic symptoms. Spinal cord injury (SCI) is an insult to the spinal cord resulting in a change, either temporary or permanent, in its normal motor, sensory, or autonomic function.

“Sciatica” is the term given to pain down the leg, which is caused by irritation of the main nerve into the leg, the sciatic nerve. This pain tends to be caused where the nerves pass through and emerge from the lower bones of the spine (lumbar vertebrae). In sciatica, there is a pain down into the leg, which travels below the knee, and may involve the foot. There may be numbness and there may be weakness of the lower leg muscles. Sciatica is usually caused by pressure on the sciatic nerve from a herniated disc (also referred to as a bulging disc, ruptured disc or pinched nerve). The problem is often diagnosed as a radiculopathy, meaning that a disc has protruded from its normal position in the vertebral column and is putting pressure on the radicular nerve (nerve root). For some people, the pain from sciatica can be severe and debilitating. For others, the pain might be infrequent and irritating, but has the potential to get worse. Usually, sciatica only affects one side, and the pain often radiates through the buttock and/or leg. Any condition that causes irritation or impingement on the sciatic nerve can cause the pain associated with sciatica. The most common cause is lumbar herniated disc. Other common causes include lumbar spinal stenosis, degenerative disc disease, or isthmic spondylolisthesis.

“Psychogenic pain” is a term that was used to refer to real physical pain that is caused by a psychological problem. It is now known as a pain disorder associated with psychological factors. Some types of mental or emotional problems can cause pain. They can also increase or prolong pain. Headaches, muscle pains, back pain, and stomach pains are some of the most common types of psychogenic pain.

“Dyesthesia” is the substitution of one sensation (usually pain) for another.

“Postherpetic neuralgia” results from herpes zoster (shingles), which causes inflammation of nerve tissue. The pain is felt as a constant deep aching or burning, as a sharp and intermittent pain, or as hypersensitivity to touch or cold. The pain may be debilitating.

“Reflex sympathetic dystrophy” (complex regional pain syndrome, type 1) and causalgia (complex regional pain syndrome, type 2) are chronic pain syndromes. They are defined as persistent burning pain accompanied by certain abnormalities that occur in the same area as the pain. Abnormalities include increased or decreased sweating, swelling, changes in skin color, and damage to the skin, hair, nails, muscle, and bone (including muscle wasting and bone loss). Both syndromes typically occur after an injury. Reflex sympathetic dystrophy results from injury to tissues other than nerve tissue (as in the shoulder-hand syndrome). Causalgia results from injury to nerve tissue.

“Temporomandibular disorders” is a collective term used to describe a number of related disorders affecting the temporomandibular joints, masticatory muscles, and associated structures, all of which have common symptoms such as pain and limited mouth opening.

“Myofacial pain” usually results from acute overstretching of muscles, whiplash, muscle fatigue, oral surgery or immobilization of muscle or as a consequence of a muscle-bracing oral habit, such as clenching and/or grinding and malocclusion. The pain experienced most often is described as a dull deep ache that is usually constant and active for months, sometimes for years. The pain is usually more intense on awakening, and is frequently unilateral or one-sided. Often, clicking or popping sounds originating from the temporomandibular joint (TMJ) area are reported. Mandibular movements are limited because patients have difficulty opening their mouth. The jaw often deviates to the affected and painful side of the face. The pain may radiate into the temple muscles and down into the sternocleidomastoid muscle and splenius capitis muscles as well as to the zygomatic process.

“Ankylosing spondylitis” (AS) is a painful, progressive, rheumatic disease. It mainly affects the spine but it can also affect other joints, tendons and ligaments. Other areas, such as the eyes, lungs, bowel and heart can also be involved. Ankylosing means fusing together. Spondylitis indicates inflammation of the vertebrae. So, AS describes the condition by which some or all of the joints and bones of the spine fuse together. Entire fusing of the spine is unusual. Many people will only have partial fusion, sometimes limited to the pelvic bones. Inflammation occurs at the site where certain ligaments or tendons attach to bone (enthesis). This is followed by some erosion of bone at the site of the attachment (enthesopathy). As the inflammation subsides, a healing process takes place and new bone develops. Movement becomes restricted where bone replaces the elastic tissue of ligaments or tendons. Repetition of this inflammatory process leads to further bone formation and the individual bones which make up your backbone, the vertebrae, can fuse together. The pelvis is commonly affected first. The lower back, chest wall and neck may also become involved at different times.

### GENERAL DESCRIPTION

Benzamides

The methods of this invention employ one or more benzamides as the active agent. These benzamides include acetamidobenzamides, aminobenzamides and nitrobenzamides such as are described by Formula I. In this formula, R₁ is a group selected from acetyl, alkyl, amino, acylamino (NHCOR₃), halo, nitro, and trifluoroalkyl, R₂ is saturated alkyl of 3 to 5 atoms, R₃ is alkyl of 1 to 5 atoms, and n is 1 or 2.

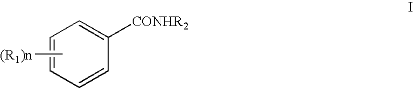

The acetamido, amino or nitro group (or groups) may be located anywhere on the ring. Thus, when n is 1 the acetamido, amino or nitro group may be at the 2, 3 or 4 position of the ring; and when n is 2 the acetamido, amino or nitro groups may be at the 2 and 3, 2 and 4, 2 and 5, 2 and 6, 3 and 4, or 3 and 5 positions of the ring. When R₂ is tert-butyl, R₁ is an acetamido group and n is 1, substitution of the acetamido group in the 3 position is not preferred.

With respect to the alkyl substituents, R′, compounds wherein R is an alkyl which does not have a hydrogen on the alpha carbon, that is, the carbon which bonds to the nitrogen of the ring, are preferred. Examples of these preferred R groups are tert-butyl and tert-amyl.

Acetamidobenzamides of Formula I of particular interest are:

N-tert-butyl-4-acetamidobenzamide (Compound A);

N-iso-propyl-4-acetamidobenzamide (Compound B);

N-tert-amyl-4-acetamidobenzamide (Compound C); and

N-cyclopropylmethyl-4-acetamidobenzamide (Compound E).

N-tert-butyl-4-acetamidobenzamide (Compound A) is the most preferred acetamidobenzamide.

Aminobenzamides and nitrobenzamides of Formula I of particular interest as active agents are:

N-iso-propyl-4-nitrobenzamide (Compound F);

N-tert-butyl-3-nitrobenzamide (Compound G);

N-tert-butyl-2-nitrobenzamide (Compound H);

N-n-butyl-4-nitrobenzamide (Compound J);

N-n-propyl-4-nitrobenzamide (Compound K);

N-tert-butyl-3,5-dinitrobenzamide (Compound L);

N-1-methylpropyl-4-nitrobenzamide (Compound M);

N-tert-butyl-4-aminobenzamide (Compound N); and

N-tert-butyl-3-aminobenzamide (Compound P).

N-tert-butyl-4-aminobenzamide (Compound N) is the most preferred of this group of materials.

When the benzamide compound contains an amino group, such as is the case with Compounds N and P, the amine functionality can be present as such or as a salt. In the salt form the amino is protonated to the cation form in combination with a pharmaceutically acceptable anion, such as chloride, bromide, iodide, nitrate, sulfonate, methane sulfonate, acetate, tartrate, oxalate, succinate, or palmoate. When these aminobenzamides are referred to it is to be understood that these salts are included as well.

Mixtures of two or more of these materials may be employed, if desired.

Pharmaceutical Compositions, Formulations and Methods of Administration

When employed in the present methods, the benzamides of this invention are typically administered in the form of pharmaceutical compositions. Such compositions can be prepared using procedures well known in the pharmaceutical art and comprise at least one active compound.

Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of this invention can be administered by any suitable routes including, by way of illustration, oral, topical, rectal, transdermal, subcutaneous, intravenous, intramuscular, intranasal, intracranial, intracerebral, intraventricular, intrathecal, and the like. Depending on the intended route of delivery, the compounds of this invention are preferably formulated as either oral, topical or injectable compositions.

Pharmaceutical compositions for oral administration can take the form of bulk liquid solutions or suspensions, or bulk powders. More commonly, however, such compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the benzamide compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. The liquid may be delivered as a spray, a paste, a gel, or a liquid drop. The desired consistency is achieved by adding in one or more hydrogels, substances that absorb water to create materials with various viscosities. Hydrogels that are suitable for use are well known in the art. See, for example, Handbook of Pharmaceutical Excipients, published by The American Pharmaceutical Association and The Pharmaceutical Society of Great Britain (1986) and the Handbook of Water-Soluble Gums and Resins, ed. By R. L. Davidson, McGraw-Hill Book Co., New York, N.Y. (1980).

Suitable hydrogels for use in the compositions include, but are not limited to, hydroxypropyl cellulose, hydroxypropyl methyl cellulose, sodium carboxymethyl cellulose and polyacrylic acid. Preferred hydrogels are cellulose ethers such as hydroxyalkylcellulose. The concentration of the hydroxycellulose used in the composition is dependent upon the particular viscosity grade used and the viscosity desired in the final product. Numerous other hydrogels are known in the art and the skilled artisan could easily ascertain the most appropriate hydrogel suitable for use in the instant invention.

The mucosal transport enhancing agents useful with the present invention facilitate the transport of the agents in the claimed invention across the mucosal membrane and into the blood stream of the patient. The mucosal transport enhancing agents are also known in the art, as noted in U.S. Pat. No. 5,284,657, incorporated herein by reference. These agents may be selected from the group of essential or volatile oils, or from non-toxic, pharmaceutically acceptable inorganic and organic acids. The essential or volatile oils may include peppermint oil, spearmint oil, menthol, eucalyptus oil, cinnamon oil, ginger oil, fennel oil, dill oil, and the like. The suitable inorganic or organic acids useful for the instant invention include but are not limited to hydrochloric acid, phosphoric acid, aromatic and aliphatic monocarboxylic or dicarboxylic acids such as acetic acid, citric acid, lactic acid, oleic acid, linoleic acid, palmitic acid, benzoic acid, salicylic acid, and other acids having similar characteristics. The term “aromatic” acid means any acid having a 6-membered ring system characteristic of benzene, whereas the term “aliphatic” acid refers to any acid having a straight chain or branched chain saturated or unsaturated hydrocarbon backbone.

Other suitable transport enhancers include anionic surfactants (e.g. sodium lauryl sulphate, sodium dodecyl sulphate), cationic surfactants (e.g. palmitoyl DL camitine chloride, cetylpyridinium chloride), nonionic surfactants (e.g. polysorbate 80, polyoxyethylene 9-lauryl ether, glyceryl monolaurate, polyoxyalkylenes, polyoxyethylene 20 cetyl ether), lipids (e.g. oleic acid), bile salts (e.g. sodium glycocholate, sodium taurocholate), and related compounds.

When the compositions and formulations of the instant invention are to be administered to the oral mucosa, the preferred pH should be in the range of pH 3 to about pH 7, with any necessary adjustments made using pharmaceutically acceptable, non-toxic buffer systems generally known in the art.

Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Topical compositions are typically formulated as a topical ointment or cream containing the active ingredient(s), generally in an amount ranging from about 0.01 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example, an oil-in-water cream base. Such topical formulations are well-known in the art and generally include additional ingredients to enhance the dermal penetration or stability of the active ingredients or the formulation. All such known topical formulations and ingredients are included within the scope of this invention.

The compounds and compositions of this invention can also be administered by a transdermal device. Accordingly, topical administration can be accomplished using a patch either of the reservoir or porous membrane type or of a solid matrix variety. Likewise, the compounds and compositions may be prepared and formulated for pulmonary delivery and can be prepared in forms (e.g. aerosol suspension, solution or the like) adapted for inhalation, in a manner well known in the art.

Injectable compositions are typically based upon injectable sterile saline or phosphate-buffered saline or other injectable carriers known in the art. As before, the benzamide compound in such compositions is typically a minor component, often being from about 0.05 to 2% by weight with the remainder being the injectable carrier and the like.

The above-described components for orally and topically administrable or injectable compositions are merely representative. Other materials as well as processing techniques and the like are set forth in Part 8 of Remington's Pharmaceutical Sciences, 18th edition, 1990, Mack Publishing Company, Easton, Pa., 18042, which is incorporated herein by reference.

The compounds of this invention can also be administered in sustained release forms or from sustained release drug delivery systems. A description of representative sustained release materials can be found in the incorporated materials in Remington's Pharmaceutical Sciences.

Benzamides may be provided in a liposome formulation. Liposome delivery has been utilized as a pharmaceutical delivery system for other compounds for a variety of applications. See, for example Langer (1990) Science 249:1527-1533; Treat et al. (1989) in Liposomes in the Therapy of Infectious Disease and Cancer, Lopez-Berestein and Fidler (eds.), Liss: New York, pp. 353-365 (1989). Many suitable liposome formulations are known to the skilled artisan, and may be employed for the purposes of the present invention. For example, see: U.S. Pat. No. 5,190,762.

In a further aspect, the compounds of the present invention in liposomes can cross the blood-brain barrier, which would allow for intravenous or oral administration. Many strategies are available for crossing the blood-brain barrier, including but not limited to, increasing the hydrophobic nature of a molecule; introducing the molecule as a conjugate to a carrier, such as transferrin, targeted to a receptor in the blood-brain barrier; and the like. In another embodiment, the molecule can be administered intracranially or intraventricularly. In yet another embodiment, a benzamide can be administered in a liposome targeted to the blood-brain barrier.

The administration of the compounds of the present invention can be alone, or in combination with other compounds effective at treating the various medical conditions contemplated by the present invention. Also, the compositions and formulations of the present invention, may be administered with a variety of analgesics, anesthetics, or anxiolytics to increase patient comfort during treatment.

The amount of a benzamide which is optimal in promoting motor or sensory improvement or reduction in pain can be determined by standard clinical techniques based on the present description. The precise dose to be employed in the formulation will also depend on the route of administration, and the seriousness of the disease or disorder, and should be decided according to the judgment of the practitioner and each subject's circumstances. Effective doses may be extrapolated from dose-response curves derived from animal model test systems.

The following formulation examples illustrate representative pharmaceutical compositions of this invention. The present invention, however, is not limited to the following pharmaceutical compositions.

Formulation 1—Tablets

A compound of formula I is admixed as a dry powder with a dry gelatin binder in an approximate 1:2 weight ratio. A minor amount of magnesium stearate is added as a lubricant. The mixture is formed into 240-270 mg tablets (80-90 mg of active benzamide compound per tablet) in a tablet press.

Formulation 2—Capsules

A compound of formula I is admixed as a dry powder with a starch diluent in an approximate 1:1 weight ratio. The mixture is filled into 250 mg capsules (125 mg of active benzamide compound per capsule).

Formulation 3—Liquid

A compound of formula I (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 ml.

Formulation 4—Injection

The compound of formula I is dissolved in a buffered sterile saline injectable aqueous medium to a concentration of approximately 5 mg/ml.

Formulation 5—Ointment

Stearyl alcohol (250 g) and white petrolatum (250 g) are melted at about 75° C. and then a mixture of a compound of formula 1 (50 g), methylparaben (0.25 g), propylparaben (0.15 g), sodium lauryl sulfate (10 g), and propylene glycol (120 g) dissolved in water (about 370 g) is added and the resulting mixture is stirred until it congeals.

Compound Utility

The compounds and pharmaceutical compositions of this invention find use as therapeutics for treating pain conditions including chronic pain, such as for example generalized pain syndrome, headache, low back and cervical pain, cancer pain, arthritis pain, neuropathic pain, psychogenic pain, and acute pain such as post operative pain, and acute traumatic injuries such as acute injury to the central nervous system (e.g. the brain and spinal cord) in mammals including humans.

The following more complete listing of pain conditions included within the definition of neuropathic pain may be found in PAIN MANAGEMENT, Rochelle Wagner and Robert R. Myers.

Examples and Causes of Neuropathic Pain

| | |
|---|---|
| Peripheral nerve trauma | Spinal cord |
| Entrapment neuropathy | Trauma, transaction, hemisection, |
| Nerve transection, including surgery | Lissauer tract section |
| Causalgia | Syrinx |
| Amputation and stump pain | Mutiple sclerosis |
| Neuroma | Tumor compression |
| Post-choracotomy pain | Arteriovenous malformation |
| Other mononeuropathies | Dyscraphism |
| Diabetic | Vitamin B12 deficiency |
| Malignant nerve/plexus invasion | Hematomyelia |
| Plexus irradiation | Syphilitic myelitis |
| Ischemic irradiation | Commissural myelotomy |
| Connective tissue disease | Brain stem |
| (rheumatoid arthritis, systemic | Wallenberg's syndrome |
| lupus erythematosus, | Multiple sclerosis |
| polyarteritis nodosa) | Tuberculoma |
| Polyneuropathies | Tumor |
| Diabetic | Syrinx |
| Alcoholic | Thalamus |
| Nutritional | Infarction |
| Amyloid | Tumor |
| Fabry disease | Surgical lesions in main |
| Chemical (e.g., anticancer therapies) | sensory necleus |
| Idiopathic | Hemorrahage |
| AIDS neuropathy | Cortical/subcortical |
| Root and dorsal root ganglion | Infarction |
| Prolapsed disk/compression | Trauma |
| Postherpetic or trigeminal neuralgia | Tumor |
| Arachnoiditis | Arteriovenous malformation |
| Root avulsion | |
| Tumor compression | |
| Surgical rhizotomy | |

Conditions Treated and Treatment Regimens

The conditions treated with the benzamide-containing compositions generally include NP and/or TBI and/or spinal cord injury, and the various symptoms which fall within the definition of all of the noted conditions. The benzamide-containing formulations can be administered to achieve a therapeutic effect. The benzamide compounds exhibit a long residency in the body. This suggests that a convenient, once-a-day regimen is possible. Other results indicate that multiple doses, such as up to three doses per day, typically, can possibly offer more effective therapy. Thus, a single dose or a multidose regimen may be used.

The benzamide-containing composition is administered in manners designed to get the drug into the patient's bloodstream. One excellent mode for accomplishing this is intravenous administration. Intravenous dose levels for treating NP range from about 0.01 mg/kg/hour of active benzamide to about 100 mg/kg/hour, all for from about 1 to about 120 hours and especially 1 to 96 hours. A preloading bolus of from about 50 to about 5000 mg may also be administered to achieve adequate steady state levels. Other forms of parenteral administration, such as intramuscular injection can be used, as well. In this case, similar dose levels are employed.

With oral dosing, one to three oral doses per day, each from about 0.1 to about 150 mg/kg of active benzamide are called for with preferred doses being from about 0.15 to about 100 mg/kg.

For the treatment of long-term conditions, such as chronic neuropathic pain, the regimen for treatment may stretch over many months or years so oral dosing is preferred for patient convenience and tolerance. With oral dosing, one to five and especially two to four and typically three oral doses per day are representative regimens. Using these dosing patterns, each dose provides from about 0.1 to about 20 mg/kg of the benzamide, with preferred doses each providing from about 0.1 to about 10 mg/kg and especially about 1 to about 5 mg/kg.

The compounds of this invention can be administered as the sole active agent or they can be administered in combination with other active agents, such as cyclooxygenase inhibitors, 5-lipoxygenase inhibitors, non-steroidal antiinflammatory drugs (NSAIDs), steroids, peripheral analgesic agents such as zomepirac, diflunisol, and the like, other active analgesic agents, such as opioid analgesic agents, and other active benzamide derivatives.

In any treatment regimen, the health care professional should assess the patient's condition and determine whether or not the patient would benefit from benzamide treatment. Some degree of experimentation to determine an optimal doing level and pattern may be needed.

A positive dose-response relationship has been observed. As such and bearing in mind the severity of the side effects and the advantages of providing maximum possible amelioration of symptoms, it may be desired in some settings to administer large amounts of benzamide such as those described above.

Methods of Preparation of Compounds

The benzamide compounds employed herein can be prepared using commonly available starting materials and readily achievable reactions. Several synthetic schemes are known and are set forth in commonly owned U.S. Pat. No. 6,194,465, the disclosure of which is incorporated herein by reference in its entirety.

The following synthetic and biological examples are offered to illustrate this invention and are not to be construed in any way as limiting the scope of this invention.

### EXAMPLES

The invention will be further described by the following Examples. Examples 1 to 19 demonstrate the preparation of acetamidobenzamides, as well as nitro- and aminobenzamides, which are representative of the benzamide compounds employed in the compositions and methods of this invention. Examples 20 to 24 present biological protocols and results illustrating the activity of the compositions of the invention in treatment of neuropathic pain (NP), traumatic brain injury (TBI) and spinal cord injury (SCI).

### EXAMPLE 1

### Preparation of N-tert-Butyl-4-aminobenzamide (Compound N)

tert-Butyl amine (14.6 g, 0.200 mole) was stirred in ethyl acetate (150 mL, purified by washing with 5% sodium carbonate solution, saturated sodium chloride solution, drying over anhydrous magnesium sulfate, and filtering through fluted filter paper) and cooled to 5° C. with an ice bath. 4-nitrobenzoyl chloride (18.6 g, 0.100 mole) in purified ethyl acetate (75 mL) was added dropwise at such a rate to maintain the temperature below 10° C. The ice bath was removed upon complete addition of benzoyl chloride solution and the reaction stirred for 4 hours. The reaction mixture was then filtered on a Büchner funnel, the filtrate washed three times with 5% HCl, once with saturated sodium chloride, dried over anhydrous magnesium sulfate, filtered through fluted filter paper, and the solvent stripped off leaving white crystalline product. The product was dried in a vacuum oven at 24 mm and 45° C. for 14 hours. This procedure produced 17.13 g of crystals of N-tert-butyl-4-nitrobenzamide (Compound Q) (77% yield), mp 162-163° C. Proton nuclear magnetic resonance (89.55 MHz in CDCl₃) showed absorptions at 8.257 ppm (d, 8.8 Hz, 2H; 3,5-aryl H); 7.878 ppm (d, 8.8 Hz, 2H; 2,6-aryl H); 6.097 ppm (bs, 1H; N—H); 1.500 ppm (s, 9H; tert-butyl H).

Palladium on carbon (5%, 75 mg) was added to Compound Q (5 g, 22.5 mmole) in 95% ethanol at 55° C. A solution of hydrazine (1.2 mL) in 95% ethanol (10 mL) was added dropwise over 30 min. and more Pd/C added (75 mg). The reaction was refluxed 3 hours, hydrazine (0.5 g) in 95% ethanol (5 mL) was added and the reaction was refluxed for another hour. The reaction was filtered on a Büchner funnel, the volume of solvent reduced under vacuum, and extracted with dichloromethane. The combined extracts were dried over magnesium sulfate and solvent stripped, leaving 3.90 g of N-tert-butyl-4-aminobenzamide (Compound N) (90% yield), melting point 125-127° C. 90 MHz proton NMR (in CDCl₃) showed absorbances at 7.290 ppm (2H, d, 8.8 Hz; 2,6 aryl H); 6.368 ppm (2H, d, 8.8 Hz; 3,5 aryl H); 5.45 ppm (1H, bs; NHC═O); 3.727 ppm (2H, bs; aryl-NH₂); 1.186 ppm (9H, s; t-butyl H).

### EXAMPLE 2

### Preparation of N-tert-Butyl-4-acetamidobenzamide (Compound A)

Acetyl chloride (0.45 g, 5.7 mmole) in ethyl acetate (25 mL) was added dropwise to Compound N (1.0 g, 5.2 mmole) and triethyl amine (0.58 g, 5.7 mmole) in ethyl acetate at 3° C. at such a rate to maintain the temperature below 10° C. The reaction was allowed to warm to room temperature, stirred 1 hour, and washed with 5% HCl. Recrystallization from acetone gave 1.08 g N-tert-butyl-4-acetamidobenzamide (Compound A)(89% yield), melting point 119-121° C. 90 MHz proton NMR (in DMSO-d₆) showed absorbances at 9.726 ppm (1H, bs, N—H); 7.715 ppm (4H, dd, 4.4 Hz; aryl H); 7.295 ppm (1H, bs; NH); 2.844 ppm (3H, s; CH₃CO); 1.448 ppm (9H, s; t-butyl H).

### EXAMPLE 3

### Preparation of N-tert-Butyl-3-nitrobenzamide (Compound G) N-tert-butyl-3-aminobenzamide (Compound P) and N-tert-butyl-3-acetamidobenzamide (Compound D)

The amidation procedures of Example 1 were followed using 3-nitrobenzoyl chloride instead of 4-nitrobenzoyl chloride. This gave N-tert-butyl-3-nitrobenzamide (Compound G) in 92% yield, melting point 123-125° C. Proton NMR (in CDCl₃) showed absorptions at 8.517 ppm (2-aryl H, s, 1H); 8.337 ppm (4-aryl H, d, 8.8 Hz, 1H); 8.121 ppm (6-aryl H, d, 6.4 Hz, 1H); 7.618 ppm (5-aryl H, m, 1H); 6.032 ppm (N—H, bs, 1H); 1.484 ppm (t-butyl H, s, 9H).

Iron (III) oxide hydroxide catalyzed hydrazine reduction produced N-tert-butyl-3-aminobenzamide (Compound P) in 53% yield, melting point 118-120° C. Proton NMR (in CDCl₃) showed absorbances at 7.088 ppm (4-6-aryl H, m, 3H); 6.794 ppm (2-aryl H, s, 1H); 5.902 ppm (N—H, bs, 1H); 3.145 ppm (aryl N—H, bs, 2H); 1.458 ppm (t-butyl H, s, 9H).

Acetylation of Compound P as described in Example 2 gave N-tert-butyl-3-acetamidobenzamide (Compound D) in 75% yield, melting point 194-195° C. Proton NMR (in CDCl₃) showed absorptions at 7.778 ppm (4-6-aryl H, m, 3H); 7.392 ppm (2-aryl H, s, 1H); 6.08 ppm (N—H, bs, 1H); 2.174 ppm (acetyl CH₃, s, 9H); 1.500 ppm (t-butyl H, s, 9H).

### EXAMPLE 4

### Preparation of N-tert-butyl-2-nitrobenzamide (Compound H) and N-tert-butyl-2-acetamidobenzamide

The method of Example 3 is repeated using 2-nitrobenzoyl chloride in the amidation step. This yields N-tert-butyl-2-nitrobenzamide (Compound H).

Reduction of the nitrobenzamide with hydrazine yields N-tert-butyl-2-aminobenzamide.

Acetylation of the aminobenzamide yields N-tert-butyl-2-acetamidobenzamide.

### EXAMPLE 5

### Preparation of N-iso-propyl-4-nitrobenzamide (Compound F) and -iso-propyl-4-acetamidobenzamide (Compound B)

The method of Example 3 is repeated using 4-nitrobenzoyl chloride and iso-propyl amine in the amidation step. This yields N-iso-propyl-4-nitrobenzamide (Compound F).

Reduction of the nitrobenzamide with hydrazine yields N-iso-propyl-4-aminobenzamide.

Acetylation of the aminobenzamide yields N-iso-propyl-4-acetamidobenzamide (Compound B).

### EXAMPLE 6

### Preparation of N-tert-amyl-4-nitrobenzamide and N-tert-amyl-4-acetamidobenzamide (Compound C)

The method of Example 3 is repeated using 4-nitrobenzoyl chloride and tert-amyl amine in the amidation step. This yields N-tert-amyl-4-nitrobenzamide.

Reduction of the nitrobenzamide with hydrazine yields N-tert-amyl-4-aminobenzamide.

Acetylation of the aminobenzamide yields N-tert-amyl-4-acetamidobenzamide (Compound C).

### EXAMPLE 7

### Preparation of N-iso-butyl-4-acetamidobenzamide

The method of Example 3 is repeated using 4-nitrobenzoyl chloride and iso-butyl amine in the amidation step. This yields N-iso-butyl-4-nitrobenzamide.

Reduction of the nitrobenzamide with hydrazine yields N-iso-butyl-4-aminobenzamide.

Acetylation of the aminobenzamide yields N-iso-butyl-4-acetamidobenzamide.

### EXAMPLE 8

### Preparation of N-n-butyl-4-nitrobenzamide (Compound J) and N-n-butyl-4-acetamidobenzamide

The method of Example 3 is repeated using 4-nitrobenzoyl chloride and n-butyl amine in the amidation step. This yields N-n-butyl-4-nitrobenzamide (Compound J).

Reduction of the nitrobenzamide with hydrazine yields N-n-butyl-4-aminobenzamide.

Acetylation of the aminobenzamide yields N-n-butyl-4-acetamidobenzamide.

### EXAMPLE 9

### Preparation of N-n-propyl-4-nitrobenzamide (Compound K) and N-n-propyl-4-acetamidobenzamide

The method of Example 3 is repeated using 4-nitrobenzoyl chloride and n-propyl amine in the amidation step. This yields N-n-propyl-4-nitrobenzamide (Compound K).

Reduction of the nitrobenzamide with hydrazine yields N-n-propyl-4-aminobenzamide.

Acetylation of the aminobenzamide yields N-n-propyl-4-acetamidobenzamide.

### EXAMPLE 10

### Preparation of N-1,2-dimethylpropyl-4-nitrobenzamide and N-1,2-dimethylipropyl-4-acetamidobenzamide

The method of Example 3 is repeated using 4-nitrobenzoyl chloride and 1,2-dimethylpropyl amine in the amidation step. This yields N-1,2-dimethylpropyl-4-nitrobenzamide.

Reduction of the nitrobenzamide with hydrazine yields N-1,2-dimethylpropyl-4-aminobenzamide.

Acetylation of the aminobenzamide yields N-1,2-dimethylpropyl-4-acetamidobenzamide.

### EXAMPLE 11

### Preparation of N-n-pentyl-4-nitrobenzamide and N-n-pentyl-4-acetamidobenzamide

The method of Example 3 is repeated using 4-nitrobenzoyl chloride and n-pentyl amine in the amidation step. This yields N-n-pentyl-4-nitrobenzamide.

Reduction of the nitrobenzamide with hydrazine yields N-n-pentyl-4-aminobenzamide.

Acetylation of the aminobenzamide yields N-n-pentyl-4-acetamidobenzamide.

### EXAMPLE 12

### Preparation of N-2-methylbutyl-4-nitrobenzamide and N-2-methylbutyl-4-acetamidobenzamide

The method of Example 3 is repeated using 4-nitrobenzoyl chloride and 2-methylbutyl amine in the amidation step. This yields N-2-methylbutyl-4-nitrobenzamide.

Reduction of the nitrobenzamide with hydrazine yields N-2-methylbutyl-4-aminobenzamide.

Acetylation of the aminobenzamide yields N-2-methylbutyl-4-acetamidobenzamide.

### EXAMPLE 13

### Preparation of N-n-pentyl-2-nitrobenzamide and N-n-pentyl-2-acetamidobenzamide

The method of Example 3 is repeated using 2-nitrobenzoyl chloride and n-pentyl amine in the amidation step. This yields N-n-pentyl-2-nitrobenzamide.

Reduction of the nitrobenzamide with hydrazine yields N-n-pentyl-2-aminobenzamide.

Acetylation of the aminobenzamide yields N-n-pentyl-2-acetamidobenzamide.

### EXAMPLE 14

### Preparation of N-tert-butyl-2,3-diacetamidobenzamide

The method of Example 3 is repeated using 2,3-dinitrobenzoyl chloride in the amidation step. This yields N-tert-butyl-2,3-dinitrobenzamide.

Reduction of the nitrobenzamide with hydrazine yields N-tert-butyl-2,3-diaminobenzamide.

Acetylation of the aminobenzamide yields N-tert-butyl-2,3-diacetamidobenzamide.

### EXAMPLE 15

### Preparation of N-tert-amyl-2,4-diacetamidobenzamide

The method of Example 3 is repeated using 2,4-dinitrobenzoyl chloride and tert-amyl amine in the amidation step. This yields N-tert-amyl-2,4-dinitrobenzamide.

Reduction of the nitrobenzamide with hydrazine yields N-tert-amyl-2,4-diaminobenzamide.

Acetylation of the aminobenzamide yields N-tert-amyl-2,4-diacetamidobenzamide.

### EXAMPLE 16

### Preparation of N-tert-butyl-2,5-diacetamidobenzamide

The method of Example 3 is repeated using 2,5-dinitrobenzoyl chloride in the amidation step. This yields N-tert-butyl-2,5-dinitrobenzamide.

Reduction of the nitrobenzamide with hydrazine yields N-tert-butyl-2,5-diaminobenzamide.

Acetylation of the aminobenzamide yields N-tert-butyl-2,5-diacetamidobenzamide.

### EXAMPLE 17

### Preparation of N-tert-butyl-2,6-diacetamidobenzamide

The method of Example 3 is repeated using 2,6-dinitrobenzoyl chloride in the amidation step. This yields N-tert-butyl-2,6-dinitrobenzamide.

Reduction of the nitrobenzamide with hydrazine yields N-tert-butyl-2,6-diaminobenzamide.

Acetylation of the aminobenzamide yields N-tert-butyl-2,6-diacetamidobenzamide.

### EXAMPLE 18

### Preparation of N-tert-butyl-3,4-diacetamidobenzamide

The method of Example 3 is repeated using 3,4-dinitrobenzoyl chloride in the amidation step. This yields N-tert-butyl-3,4-dinitrobenzamide.

Reduction of the nitrobenzamide with hydrazine yields N-tert-butyl-3,4-diaminobenzamide.

Acetylation of the aminobenzamide yields N-tert-butyl-3,4-diacetamidobenzamide.

### EXAMPLE 19

### Preparation of N-tert-butyl-3,5-diacetamidobenzamide

The method of Example 3 is repeated using 3,5-dinitrobenzoyl chloride in the amidation step. This yields N-tert-butyl-3,5-dinitrobenzamide.

Reduction of the nitrobenzamide with hydrazine yields N-tert-butyl-3,5-diaminobenzamide.

Acetylation of the aminobenzamide yields N-tert-butyl-3,5-diacetamidobenzamide.

### Biological Experiments

Compounds of the invention were tested for pharmacokinetic properties and therapeutic activity. In particular, a group of tests looked at pharmacokinetic issues such as clearance rate, while the remaining tests assessed the effects of certain compounds on specific biological parameters in animal models of neuropathic pain, traumatic brain injury and spinal cord injury. The protocol and results follow below.

### EXAMPLE 20

Bioavailability of Compounds A and N

The absolute oral bioavailability of Compound A was determined by comparing the area-under-the-curve (“AUC”) following a 20 mg/kg IV dose of Compound A (2 ml/kg, 75% PEG solution) to a 20 mg/kg dose of Compound A dissolved in 1% methyl cellulose. Blood concentrations were determined at 0, 0.083, 0.15, 0.5, 1, 2, 4, 8, and 24 hours post-IV dose and 0, 0.5, 1, 2, 4 and 8 hours post-oral dose. Four animals were dosed orally and four animals were dosed intravenously.

In another experiment, six rats were orally dosed with 30 mg/kg of Compound A dissolved in 1% methyl cellulose. Blood samples were taken at 0.5, 1, 2, 5, 8, 12, 16, 20 and 24 hours. The apparent t_{1/2} for Compound A in the blood was 8 hours. This is a very long t_{1/2} for a drug in rats and is a good predictor of once-a-day dosing for Compound A in humans. Such a dosing regimen would be a significant therapeutic advantage in the clinic.

The blood samples were analyzed by HPLC. Detection was by UV at 262 nm. Responses were converted to concentrations by comparison to responses from calibration standards injected onto the column. The resulting concentrations were used to calculate the AUC. Through oral dosing, there was an absolute bioavailability of 52% of Compound A.

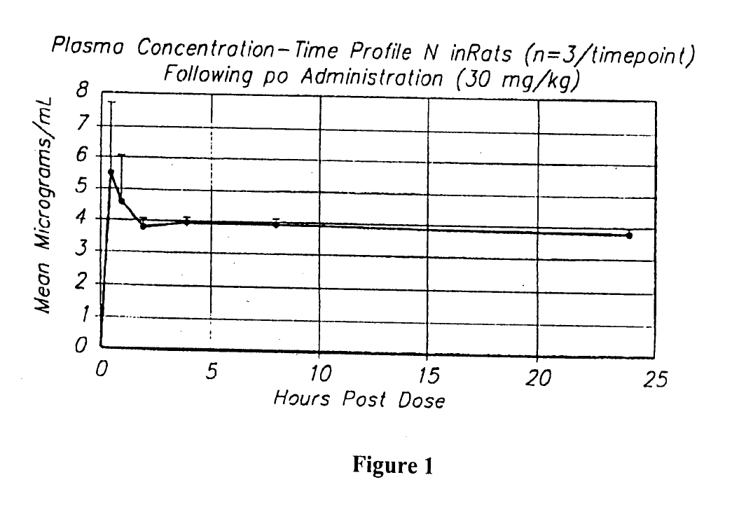

An additional series of bioavailability experiments were conducted using compounds A and N. These studies confirmed the excellent bioavailability of compound A and demonstrated similar results for compound N. The results are given in Table 1. The time course of plasma concentration of compound N is depicted in FIG. 1 as well.

TABLE 1
PK Parameter Estimates for Most Active NRTs from Screening
(NRTs Dosed 30 mg/kg po in 1% Methyl Cellulose,
n = 3 rats/NRT)

| Compound | A | N |
|---|---|---|
| Cₘₐₓ (μg/mL) | 34 | 5.4 |
| Tₘₐₓ (h) | 8.0 | 0.5 |
| AUC_{0-∞} (μg · hr/mL) | 745 | 1026 |
| Elim.t_{1/2}(h) | 11 | 355 |

As FIG. 1 shows, for Compound N, Tₘₐₓ occurs early at 0.5 h, and, although Cₘₐₓ is somewhat lower than that found following dosing with Compound A, the lower clearance of Compound N results in an overall greater systemic exposure for a given dose for Compound N than for Compound A. In addition, the extremely long terminal half-life observed for Compound N may partially explain the extraordinary efficacy of the compound because the half-life permits the presence of Compound N following a single dose at a substantial concentration throughout the complete multi-day inflammatory process. The exceptionally long t_{1/2} of Compound N in rats predicts possibly weekly dosing with Compound N. In this regard, note the relatively low variability in plasma concentration of Compound N once the absorption and initial elimination phase are complete.

### EXAMPLE 21

Animal Models for Neuropathic Pain, Traumatic Brain Injury and Spinal Cord Injury

General Considerations in Development of the Neuropathic Pain Animal Models

Taxol is a chemotherapeutic reagent for treatment of cancer patients (Polomano, R C et al. (2001) Pain, 94: 293-304; Wang J et al. (2002) Br J Haematol 118: 638-645; Dina O A et al. (2001) Neuroscience 108: 507-515; Asakuma J et al. (2003) Cancer Res. 63(6): 1365-1370). Cancer patients under Taxol chemotherapy can develop neuropathic pain, with a symptom of severe mechanical allodynia, so that even a light touch (non-noxious stimulus under normal conditions) becomes painful. Taxol has been shown to interact with beta-tubulin, and hence functions as a microtubule stabilizer. Recent studies show that Taxol is involved in TNF-alpha release in microglial cells (Wang J et al. (2002) Br J Haematol 118: 638-645; Asakuma J et al. (2003) Cancer Res. 15; 63: 1365-1370). Due to its clinical relevance, this model for Taxol-induced neuropathic pain was developed in mice and rats. It is believed that small-diameter high threshold nociceptive C-fibers in the peripheral sensory nervous systems are largely, if not exclusively, affected in the Taxol-induced neuropathic pain model (Polomano, R C et al. (2001) Pain, 94: 293-304; Dina O A et al. (2001) Neuroscience 108: 507-515). The Taxol model was utilized to assess the analgesic effects of Compound A.

Materials and Methods

Test and control articles

Compound A (Lot number XX) was synthesized by Centaur. HPLC and Mass-spectromery analysis showed 99% purity. Gabapentine, as a positive control, was purchased from Tocris Cookson Inc.

Formulation(s)

PEG-200 formulation for i.p. administration of Compound A: PEG-200 was diluted in deionized destilled water to give a final concentration of 75% (volume/volume). Compound A was weighed to yield a final concentration of 10 mg/ml in 75% PEG-200 vehicle and sonicated for 12×30 seconds with 5 second intervals on ice to generate a Compound A suspension. 100 μL of either Compound A suspension or control vehicle (75% PEG-200) per 20 grams of mice (body weight) was administered via i.p., thus yielding a dose of 50 mg/kg (body weight).

Cremophor EL/alcohol/saline formulation for i.p. administration of Compound A: 40 mg/ml, 20 mg/ml or 10 mg/ml of Compound A was dissolved in Cremophor EL/alcohol (90:10) (volume:volume), respectively, by brief sonication on ice, then freshly diluted 10-fold in saline solution (9% Cremophor EL; 1% ETOH and 90% saline) to give a final concentration of 4 mg/ml, 2 mg/ml and 1 mg/ml, respectively, before injection. 100 μL of Compound A solution at a concentration of 5, 2, and 1 mg/ml was administered via i.p. to mice (per 20 grams of body weight), thus yielding a dose of 20 mg/kg (body weight), 10 mg/kg (body weight), and 5 mg/kg (body weight), respectively. The control vehicle was 9% Cremophor EL/1% ETOH/90% saline.

Methylcellulose formulation for oral gavage feeding of Compound A in rats: 1% of methylcellulose was dissolved in water and kept at 4° C. 50 mg/ml and 20 mg/ml of Compound A/1% methylcellulose suspension was made by 12×30 seconds sonication with 5 second intervals on ice. 1 ml per kg of body weight of either 50 mg/ml or 20 mg/ml of Compound A was administered to rats via oral gavage feeding (p.o.), to yield dose of either 50 mg/Kg (body weight) or 20 mg/Kg (body weight). The same corresponding volume of 1% methylcellulose was used as a vehicle control.

Formulation of Taxol for i.p. administration (Simmons Z (2002), Curr Opin Neurol 15: 595-603). For use in the Taxol-induced neuropathic pain mouse model, 1 mg of Taxol was dissolved in 50% of Cremophor EL/50% of absolute ETOH (volume/volume), and kept in the dark at 4° C. no longer than 3 days. Prior to administration, the Taxol solution was diluted with saline (1 volume of 1 mg taxol solution in Cremophor EL/ETOH (50:50) was added with 4 volumes of saline) to give 0.2 mg/ml of taxol in 10% Cremophor EL, 10% ETOH and 80% of saline. 100 μL of taxol solution per 20 grams body weight of mouse was administered to mouse via i.p. to give a dose of 1 mg/kg (body weight). For use in the Taxol-induced neuropathic pain rat model, 5 mg of Taxol was dissolved in 50% of Cremophor EL/50% of absolute ETOH (volume/volume), and kept in the dark at 4° C. no longer than 3 days. Prior to administration, the Taxol solution was diluted with saline (1 volume of 1 mg taxol solution in Cremophor EL/ETOH (50:50) was added with 4 volumes of saline) to give 1 mg/ml of Taxol in 10% Cremorphor EL, 10% ETOH and 80% of saline. 200 μL of Taxol solution per 200 grams body weight of rat was administered to mouse via i.p. to give a dose of 1 mg/kg (body weight).

Test System

The use of laboratory animals was under protocol REN-6, which was approved by IACUC. For mouse neuropathic pain studies, five-week old male C57B16 mice were obtained from Charles River, San Diego, Calif. A total number of 120 mice was used for the studies. 4 mice per cage were housed under standard conditions with 12-hour light and 12-hour dark cycles (in Renovis, Inc, South San Francisco facility). For rat neuropathic pain studies, Sprague-Dawley male rats, weighing 150-170 grams, were obtained from Charles River, San Diego, Calif. A total number of 42 rats were used for this study. 3 rats per cage were housed under standard conditions with 12-hour light and 12-hour dark cycles (Renovis, Inc. Redwood City facility).

Equipment

von Frey Filament sets were purchased from Stoeling. Customized mouse enclosures [with a dimension of 2.5″×2.5″×3″ (W×H×L)] and rat enclosures [with a dimension of 3.5″×4″×10″ (W×H×L)] were ordered from IITC for mechanical allodynia tests.

Reagents

Taxol was purchased from ICN; Cremophor EL was purchased from Sigma-Aldrich; Compound A (Lot number XX) was synthesized by Centaur; PEG-200 was purchased from Sigma-Aldrich, Methylcellulose was purchased from Sigma-Aldrich; Gabapentine was purchased from Tocris Cookson Inc.

Taxol-Induced Neuropathic Pain Model in Mice

C57B16 male mice were preconditioned (trained) in the enclosure one hour per day in the morning for one week prior to the baseline measurement. The baseline response to the mechanical stimulus was measured using a von Frey filament with 0.6 gram of force (von Frey number 3.84). Briefly, the von Frey filament, was applied to the plantar area of the right hind paw of mouse. The frequency of paw withdrawal was calculated in terms of percentage response, where the higher percentage indicates more pain, whereas the lower the percentage indicates less pain. Based on past experience with the C57B16 strain of adult male mice, animals with baseline no greater than 40% of response were included in further studies, while those with greater than 40% of paw withdrawal response to this particular force of stimulus, known as spontaneous mechanical allodynia, were discarded from these studies. The paw withdrawal behavior includes, licking the stimulated paw, vigorously shaking the paw and active avoidance upon application of innocuous mechanical stimuli. For those mice without spontaneous mechanical allodynia, Taxol was administered to the mice daily, 5 days a week, for longer than a one week period. Generally, mice treated with daily i.p. administration of 1 mg/kg (body weight) of Taxol develop relatively stable mechanical allodynia, neuropathic pain, on day 9 and thereafter. Prior to compound A administration, the responses of mice to 0.6 gram of von Frey filament were measured for two consecutive days in the morning. Only those mice with paw withdrawal response to this particular von Frey filament stimulus no less than 60%, counted as mechanical allodynia, were included for later testing with the compounds of interest.

Taxol-Induced Neuropathic Pain in a Rat Model

Sprague-Dawley male rats with 150-160 grams of body weight were used in the studies presented herein. Rats were trained in the enclosure one hour per day in the morning for a one week period prior to obtaining the baseline response. Whereas the mouse studies assessed percentage of response, the rat studies determined 50% of the paw withdrawal threshold, also called the up-down method as developed by Chung and colleagues. In order to obtain consistent data, naïve rats with paw withdrawal threshold no less than 4 grams were included for further studies. Taxol at a dose of 1 mg/kg (body weight) was administered daily to rats intraperitoneally. As observed in mice, the rats treated with Taxol daily developed stable mechanical allodynia on day 9 and thereafter. On day 13 and day 14 of Taxol induction, the rats pain behavior in term of 50% of paw withdrawal threshold was measured. Only those rats with paw withdrawal threshold measurements no greater than 7 grams for two consecutive days were included for drug testing. Generally speaking, the mechanical allodynia in Taxol treated rats fell in the range of 1-4 gram of paw withdrawal threshold.

A total of five studies were performed in the rodent Taxol models for assessment of different formulations and different dosing regimens of Compound A in treatment of neuropathic pain. These are summarized below.

Initial Study in the Mouse Model of Neuropathic Pain Using a Single Dose of Compound A in PEG-200.

Five-week old, male C57B1/6 mice were purchased from Charles River and housed at the Renovis animal facility in South San Francisco. Mice were trained for one week in an enclosure made of Plexiglas with dimensions of 2 in×2.5 in×3.5 in (W, H, and L) with a meshed metal support. The baseline of response to mechanical stimulus was measured using a von Frey Filament (0.6 g=3.84 mN). Normally, the baseline response is between 0 to 40%.

Taxol was dissolved (1 mg/ml) in 50% Cremophor EL and 50% alcohol, and freshly diluted with saline to the final concentration of 0.2 mg/ml in 10% Cremophor EL, 10% alcohol. Mice were administered Taxol at 1 mg/kg of body weight, via ip daily, while the animals of the control group received vehicle (10% Cremophor EL and 10% alcohol in saline).

Five-week old C57B1/6 male mice were trained for one week in the von Frey filament test, and baseline response (percent paw withdrawal over 40 tests) to a von Frey filament (0.6 gram=3.84 mN) was measured (average shown in the graph as “baseline (mean)”=31%).

Trained mice were treated with 1 mg/kg (body weight) taxol in 10% Cremophor EL/10% ethanol via ip injection daily through the end of the experiment in order to induce the neuropathic pain state.

The percent pain response (percent paw withdrawal over 40 tests) with a von Frey filament (0.6 gram=3.84 mN) was measured at day 8 and day 9 after pain induction (average of these two shown in the graph as “pre-dosing (mean)”=75%). Those animals with either sensitive baseline (>40% response) or without pain (<60% response) after 9 days of taxol injection were not included in the data analysis. Animals receiving this daily dose of Taxol will develop neuropathic pain.

Preparation of Compound A Solution

A quantity of 200 mg of compound A (Example 2) was dissolved in 0.4 ml of 100% DMSO to yield a concentration of 500 mg/ml, and was kept at 4° C. until use. Methylcellulose was dissolved in deionized-distilled water to yield a concentration of 1%. The freshly prepared meythlcellulose was used to make a suspension of the compound, 10 mg of compound in 2.5% DMSO (V/V) and 1% methylcellulose. The suspension was sonicated for 6 min with a 5 second interval every 30 seconds before administration to animals.

Compound Administration and Behavior Measurements

A quantity of 50 mg/kg (body weight) compound or vehicle (75% polyethylene glycol (PEG)), 100 μl/kg (body weight) was given to animals via i.p. injection in a randomized, blinded fashion. The animals were then placed in the enclosure for assay. Mechanical allodynia was measured with von Frey filament (0.6 g=3.84 mN) at various times after administration.

Results: Compound A Inhibits Mechanical Allodynia in Neuropathic Pain Model

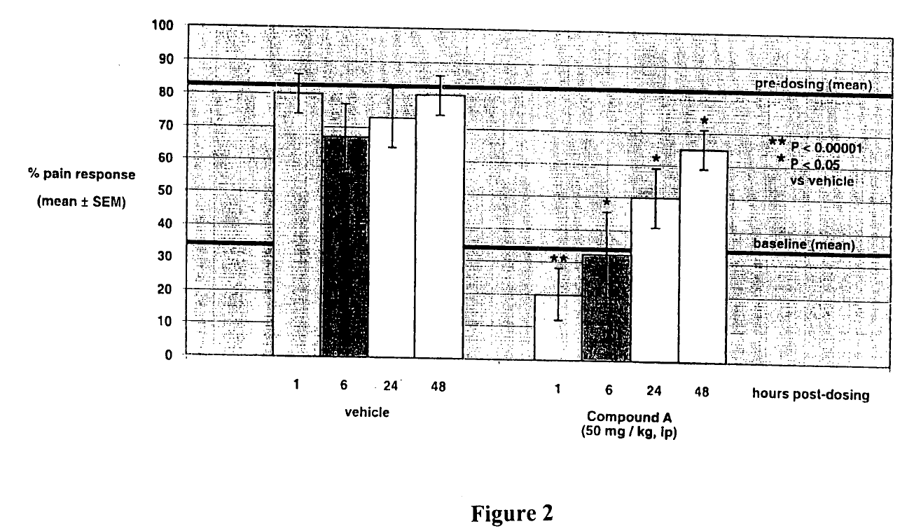

Nine taxol-treated animals received 100 μl/kg (body weight) vehicle (75% polyethylene glycol (PEG)) via ip injection; 8 taxol-treated animals were injected ip with a suspension of Compound A in vehicle at dose of 50 mg/kg (body weight). The percent pain response (percent paw withdrawal over 40 tests) with a von Frey filament (0.6 gram=3.84 mN) was measured at 1, 6, 24, and 48 hours after the single dose of either vehicle or Compound A. The results, in terms of the average pain response for each group (+SEM), are shown in FIG. 2. P-values at each time point for Compound A responses vs. vehicle-treated animal responses were based on two sample, one-tailed t tests assuming unequal variances. The higher the percentage the greater the inferred pain.

Additional Single Dose Efficacy Study of Compound A in the Taxol-Induced Neuropathic Pain Mouse Model Using a Peg-200 Formulation

Dosing Regimen:

24 adult male C57B16 mice were employed for this study. 17 animals who met the criteria of baseline less than 40% of response and response greater than 60% after Taxol treatment on day 7 and day 9, were used for assessing the analgesic effect of Compound A. This batch of mice had baseline responses of 30% on average and had a response of 80% on day seven and 75% on day nine of Taxol treatment on average. The mice were divided into two groups in a randomized fashion. One group containing 9 mice received 100 μL of 75% PEG-200 as vehicle control, the other group of 8 mice received 100 μL of Compound A at 50 mg/kg (body weight). The pain behavior measurements were blinded. Briefly, the testing mice were weighed and given the proper dose of Compound A (or vehicle), then habituated in the enclosure before mechanical allodynia measurements were made. The mechanical allodynia of each individual testing mouse was measured at 1 hour, 6 hour, 24 hour and 48 hour post dosing. After finishing the last time point measurement, the study was unblinded and the data of percentage response to von Frey filament stimuli were analyzed.

Results:

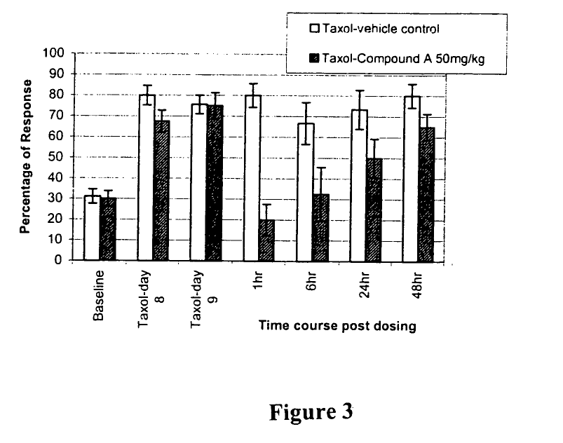

As shown in FIG. 3, animals receiving Compound A at a dose of 50 mg/kg of body weight responded to 0.6 gram of von Frey filament stimulus: 20+/−7.6% (+1-SEM) at 1-hour postdosing; 32.5+/−13.1% at 2-hour postdosing; 50+/−9.3% at 24-hour postdosing and 65+/−6.3% at 48-hour postdosing animals treated with Compound A, while the mice receiving vehicle control gave a response of 80+/−5.8% at 1-hour postdosing, 66.7+/−10.00% at 6-hour postdosing, 73+/−9.4% at 24-hour postdosing and 80+/−5.8% at 48-hour post dosing. Statistical analysis using a one-tailed t-test gave a p-value of 9.65E-6, 0.0283, 0.0489 and 0.0494, respectively, at the corresponding postdosing time points. This study indicated that Compound A had a strong analgesic effect in the Taxol-induced neuropathic pain mouse model.

Dose-Response Study of Compound A in the Taxol-Induced Neuropathic Pain Mouse Model Using Cremophor EL/ETOH/Saline Formulation

Dosing Regimen:

The initial efficacy results of Compound A for the neuropathic pain indication in the Taxol mouse model prompted further investigation to determine the dose response and pain killing effects of Compound A using a chronic dosing regimen. For these studies, a new formulation was developed, using 9% Cremophor EL, 1% ETOH and 90% saline as vehicle for intraperitoneal administration. The rationale for development of this formulation is to confirm the following: 1) That the analgesic effect of Compound A is not due to PEG-200 plus Compound A; 2) Compound A is not soluble in 75% PEG-200, rather a suspension, hence affecting the drug efficacy, while Compound A is quite soluble in 9% Cremophor EL, 1% ETOH and 90% saline;

3) 75% PEG-200 vehicle is not a good choice for a chronic dosing study since it is toxic to mice; 4) Taxol was dissolved in Cremophor EL/ETOH/saline solution, thus using 9% Cremophor EL, 1% ETOH and 90% saline as a vehicle for chronic dosing of Compound A will have less interference with daily Taxol treatment. 100 μL of Compound A solution at concentrations of 5, 2, and 1 mg/ml was administered intraperitoneally to mice (per 20 grams of body weight), thus yielding a dose of 20 mg/kg (body weight), 10 mg/kg (body weight), and 5 mg/kg (body weight), respectively. The control vehicle was 9% Cremophor EL/1% ETOH/90% saline, and 100 μL of vehicle was administered intraperitoneally to mice (per 20 grams of body weight). 48 adult male C57B16 mice were used for this study. The baseline and pain behavior measurements were as indicated above. After 21-days of Taxol treatment, 39 mice met criteria, as mentioned above, and were therefore included for Compound A testing. The baseline of this batch of mice was 12.8+/−2.4% (+/SEM), and the pain response was 77.2+/−2.6% on day 21 of Taxol treatment. On day 22, mice were randomly assigned into four groups: 9 mice were given vehicle; 10 mice were given 5 mg/kg (body weight) of Compound A; 10 mice were given 10 mg/kg and 10 mice were given 20 mg/kg. The behavior measurements were in a blind fashion at 1-hour, 3-hour, 6-hour, 24-hour postdosing time points.

Results:

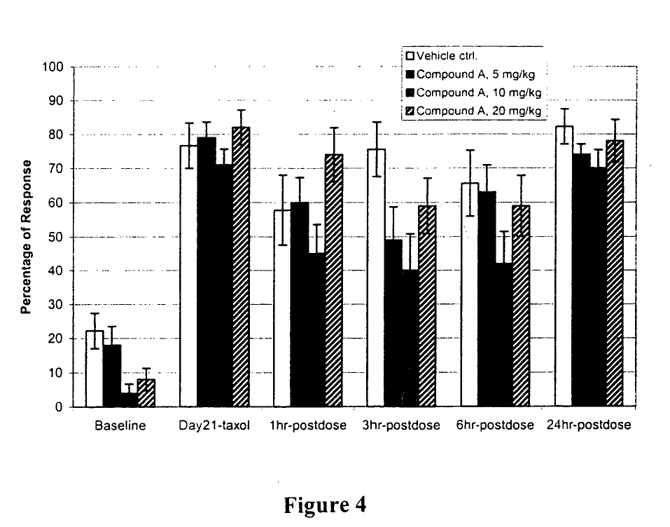

As shown in FIG. 4, the dose response study demonstrated that Compound A had an analgesic effect at 5 mg and 10 mg/kg (body weight) in a dose-dependent manner and reached peak efficacy at 3-hour post dosing time points; the numbers of percentage responses are 49+/9.7% (+/−SEM, p-value equals 0.0025) at 5 mg/kg dose and 40+/−10.9% (+/SEM, p-value equals 0.0009) at 10 mg/kg dose in comparison with vehicle treated animals, 75.6+/8.0% (+/−SEM). The 10 mg/kg dose showed pain blocking efficacy at 6-hour postdosing with a percentage of response of 42+/−95% (+/−SEM, p-value equals to 0.00496) in comparison to the vehicle treated group (65.6+/−9.6%). The group receiving 20 mg/kg dose also showed attenuated mechanical allodynia at 3-hours postdosing (59+/−8.1%), however, this group did not reach statistical significance (p-value equals 0.08).

Chronic Dosing Study of Compound A in Taxol-Induced Neuropathic Pain Mouse Model Using Cremophor EL/ETOH/Saline Formulation

Dosing Regimen:

48 adult male C57B16 mice were used for this study. The baseline and pain behavior measurements were as indicated above. After 21-days of Taxol treatment, 36 mice met criteria, as mentioned above, and were therefore included for Compound A testing. The baseline of this batch of mice was 12.8+/−2.4% (+/SEM), and the pain response was 77.2+/−2.6% on day 21 and day 23 of Taxol treatment. On day 24, mice were randomly assigned into three groups: 12 mice were given vehicle; 12 mice were given 5 mg/kg (body weight) of Compound A; and 12 mice were given 10 mg/kg (body weight) of Compound A. The dosing of mice was carried out in the morning, one dose per day for 14 days (the Taxol treatments were continued throughout the course, in the late afternoon). The behavior measurements were performed once in the early morning prior to Compound A dosing and once 3 hours after Compound A dosing. 100 μL solutions of Compound A at concentrations of 2 and 1 mg/ml, were administered intraperitoneally to mice (per 20 grams of body weight), thus yielding doses of 10 mg/kg (body weight)/per day, and 5 mg/kg (body weight/per day, respectively. The control vehicle was 9% Cremophor EL/1% ETOH/90% saline and 100 μL of vehicle was administered via i.p. to mice (per 20 grams of body weight, per day). The dosing of animals was carried out daily, one dose per day for 14 days.

Results:

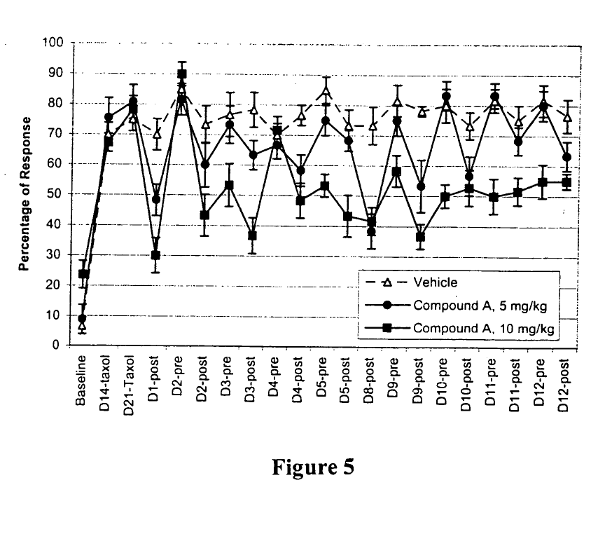

As shown in FIG. 5, the chronic dosing study revealed that animals receiving 5 mg/kg/per day and 10 mg/kg/per day showed less mechanical allodynia than those of the vehicle control group. The animal given 10 mg/kg/per day of Compound A experienced much less pain. The chronic dosing of Compound A showed no significant drug tolerance or drug accumulation effects.

Dose-Response Study of Compound A in the Taxol-Induced Neuropathic Pain model in rats using a 1% methylcellulose formulation

Dosing Regimen:

1 ml per kg of body weight of either 50 mg/ml or 20 mg/ml of Compound A was administered to rats via oral gavage feeding (p.o.), to yield a dose of either 50 mg/Kg (body weight) or 20 mg/Kg (body weight). The same corresponding volume of 1% methylcellulose was used as vehicle control. 34 male Sprague-Dawely rats were employed for the study. The baseline paw withdrawal threshold (grams of force applied to the plantar area of the right hind paw) and neuropathic pain induction with daily intraperitoneal administration of 1 mg/kg (body weight) was performed as indicated in the previous section. The Taxol treatment was continued for 15 days until the pain threshold remained relatively stable. Animals with baseline greater than 4 grams and with threshold not less than 7 grams post induction were included in the drug testing. 27 rats met the criteria for the next step studies and were randomly assigned into four groups. Group 1 consisting of 7 rats was given 1% methylcellulose as vehicle control group, 1 ml of vehicle per kilograms of body weight was administered to animal via p.o.; group 2 with 6 animals was dosed at 20 mg/kg (body weight) of Compound A via oral gavage feeding (p.o.); group 3 with 7 rats was dosed at 50 mg/kg of Compound A, and the fourth group with 7 rats received Gabapentine at 50 mg/kg via p.o. in the same vehicle as a positive control group. Those rats were habituated in the enclosure and 50% of paw withdrawal thresholds (grams of force) were measured using a set of von Frey filaments, as described in the previous section, at time points of 1, 2, 3, 6, and 24 hours postdosing in a blinded fashion. The tester had no knowledge of which animals received drug or vehicle. It was noted that rats tested were very sleepy in the late afternoon (for 6 hour postdosing measurement), thus the data would not be reliable for 6-hour postdosing time points. It was also noted that one of the rats had a bleeding left paw during the period of 6-hour postdosing test, thus the data obtained from this animal at 6 hour postdosing time point was discarded. After finishing the 24 hour postdosing measurement, the paw withdrawal threshold of each animal was calculated and data was analyzed.

Results:

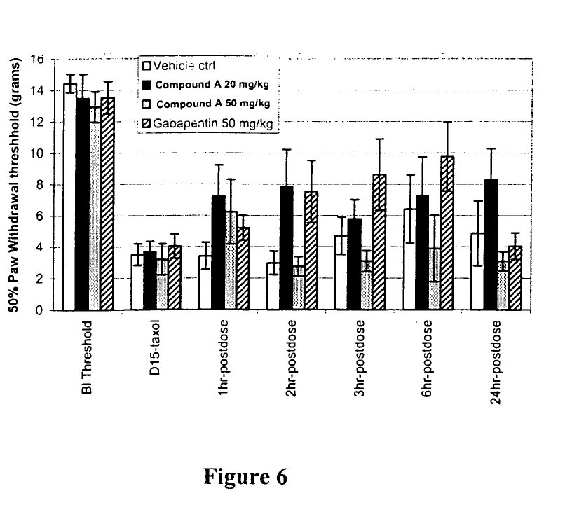

As shown in FIG. 6 the results demonstrate that Compound A has an analgesic effect on neuropathic pain at 20 mg/kg dose. The effects showed a fast onset (one-hour postdosing) and reached peak at 2 hour postdosing time points, and remained at similar levels during the testing period, and increased the paw withdrawal threshold to greater than 7 grams as compared to the rats treated with vehicle (about 4 grams of threshold). The analgesic effect of Compound A was comparable to the positive control, Gabapentine (at 50 mg/kg dose). The analgesic effect of Gabapentine reached peak at 3-6 hours post dosing. Compound A at 50 mg/kg dose showed a pain blocking effect at 1 hour postdosing, with the effect diminished later on. The difference in dose-dependency between rats and mice may be due to either the difference of formulation for treating the mice versus the rats or due to the species difference or both.

### EXAMPLE 22

Traumatic Brain Injury Model

Compound A was tested for efficacy in an animal model of traumatic brain injury. Specifically, Compound A was evaluated in the lateral fluid percussion injury (FPI) model developed by McIntosh (McIntosh T. K., et. al (1989), Neuroscience 28:233-244). This model replicates many of the pathological aspects found in humans who have sustained traumatic brain injury. Furthermore, the damage caused by the injury is sensitive to pharmacological intervention, and thus provides a robust method for preclinical efficacy testing. In this model, animals are subjected to a fluid percussion brain injury, and are then given Compound A shortly thereafter. At specific time points, animals are evaluated using outcome measures that assess cognitive and locomoter deficits that are consequences of the injury. In addition, following behavioral analysis, animals are sacrificed and their brains assessed with respect to a variety of histopathological parameters. A comparison is then made between Compound A-treated and untreated animals to determine if the compound can attenuate the behavioral deficits and histological derangements normally caused by Fluid Percussion Injury.

Experimental Protocol

For these studies, experimental parameters are used that were previously developed by Dr. Tracy McIntosh, Director of the University of Pennsylvania Head Injury Center. In these studies, male rats (350-400 g) are used for injuries. All animals are injured at a moderate/severe injury level (2.5-3.0 atm) via a craniotomy positioned over the left parietal cortex centered between Bregma and Lambda. Fifteen minutes after injury, they are randomly assigned into groups and treated with either a compound of the invention such as Compound A, or vehicle. Compound A may be formulated in either 75% PEG 200 or in 50% PEG 400 and 25% ethanol and doses of about 10 to 20 mg/kg/BID may be administered. The total amount administered per day should range from about 20 to 40 mg/kg. Compound or vehicle are delivered via IP injection 15 minutes after injury, followed by oral gavage or IP injection b.i.d. for up to 5 to 7 days following the injury. Animals are assigned to one of 2 study groups. Animals in the first group are tested for memory deficits 48 hrs post injury, and then sacrificed for acute analysis of Edema (Brain water content). Animals in the second group are tested for locomotor deficits and learning deficits at a later time point. Following behavioral testing, these animals are sacrificed, and their brains are analyzed for tissue damage (cortical volume loss, and neuronal cell counts), and other histopathological readouts.

Group 1—Analysis of Post-Trauma Cognitive Deficits and Brain Water Content (Compound A=12-20 Animals, Vehicle=12-20 Animals)

Cognitive Deficits

The ability of Compound A to restore cognitive deficits in spatial memory and learning is evaluated at 48 hrs post injury using the Morris watermaze paradigm (Smith D. H. et al. (1991), J. Neurotrauma 8(4): 259-269; Schmidt, R. H. et al. (1999), J. Neurotrauma 16(12): 1139-1147). In this assay, animals are trained to swim in a 1 m or 2 m circular water pool containing a submerged platform. Animals learn to orient themselves in the pool and to locate the platform by using distant visual cues placed on the walls of the room. In the learning paradigm, the animals are trained to find the platform, and the reduction in latency with increasing number of training sessions is recorded. In the memory paradigm, animals are given 20 training runs over a 2 day period prior to the injury. Following the second training session, the injury is delivered. 48 hours after injury, animals are tested for memory function by placing them into the pool with the platform removed, and recording the time spent swimming in defined areas of the pool. A significant memory score is generated if the animal spends more time swimming in areas where the platform was or close to where the platform was than in the other areas of the pool.

To analyze e.g. Compound A, animals are tested in the memory paradigm. Animals from Compound A and vehicle treatment groups are trained to swim to the submerged platform over a 2 day period (10 swims/day). Following training, animals are injured, and treated with Compound A via IP injection 15 minutes following injury. For the following 48 hours, they receive Compound A twice daily via oral gavage or IP injection. 48 hrs following injury, they are tested in the memory paradigm.

This assay is a very useful test of cognitive function because it is sensitive to degree of injury, and is responsive to pharmacological interventions.

Brain Water Content

Following water maze analysis, animals are euthanized, and their brains dissected. Brains are chilled on a frozen block, and a 3-5 mm coronal section surrounding the injury site is dissected. This section is subdissected into the following regions: left parietal cortex (injury region), contralateral parietal cortex (control), parietal cortex adjacent to the injury region (left and right), and left and right hippocampal regions. Tissue pieces are weighed, and then dried overnight at 100° C.

Cerebral edema is the percent of tissue weight contributed by water, and is calculated by the formula:

% water=(wet weight−dry weight)/(wet weight)*100

In this manner, the ability of the compounds used in the invention to attenuate posttraumatic cerebral edema is evaluated.

Group 2—Analysis of Post-Trauma Neurological Deficits and Histological (12-20 Animals to Receive Test Compound and 12-20 Animals Receive Vehicle)

Neurological Evaluation

Neurological testing is used to assess the ability of the compounds of the invention, such as Compound A, to attenuate the locomoter deficits normally induced by the Fluid Percussion Injury. Testing is begun 24 hr after the injury, and continues weekly for 1-4 weeks depending on the outcomes observed. Locomoter analysis is based on a set of tests that primarily assess locomoter and vestibulomotor function. This analysis includes a composite neuroscore, that largely involve tests of reflexive locomoter function. Additional tests include the beam balance (Scherbel, U. et al. (1999), Proc. Natl. Acad. Sci. 96: 8721-8726) and the rotating pole (Mattiasson, G. V. et al. (2000), J. Neuroscience Methods 95: 75-82), which test coordination and vestibulomotor function. The composite neuroscore evaluates the following behaviors: 1) contralateral forelimb flexion upon suspension by the tail; 2) hindlimb flexion upon suspension by the tail; 3) resistance to lateral pulsion to the left and to the right; 4) Ability to stand on an inclined angle board. Animals are evaluated on the their ability to execute the appropriate movements as well as their strength and coordination. They are graded on a 5 point scale with Grade 4 representing normal behavior and Grade 0 representing a severe deficit. The combined neuroscore is the sum of scores from the 4 tests.

In addition to the neuroscore, vestibulomoter function is evaluated using the rotating pole and the balance beam. On the balance beam, animals are trained to traverse a 2 cm wooden beam. After training, they are placed on the beam and tested. They are evaluated for ability to traverse the beam in a normal fashion, coordination of movements, and foot slips, and as in the neuroscore, are given a score from 0-4 depending on the degree of deficit. The rotating pole is an approximately 2 m pole that can rotate in either a clockwise or counterclockwise direction. Prior to evaluation, the animal is trained to traverse the non-rotating pole from one end to other. For testing, the pole will be set to rotate at a constant speed, and the animal is then placed onto the pole. Latency to traverse the pole, as well as foot slips and/or falls is evaluated. Animals are scored on a 5 point scale with 0 indicating the greatest deficit.

Taken together, these scores of locomoter activity provide a sensitive and accurate assessment of severity of injury.

Histopathological Analysis

Subsequent to neurological analysis, brain tissue from the animals is prepared for histological analysis. Animals are euthanized, and then transcardially perfused with 4% Paraformaldehyde (PFA). Their brains are dissected, and post fixed in 4% PFA, cryoprotected in 30% sucrose, and then frozen for sectioning. Serial coronal sections from the injury region are cut and stained with Hematoxylin and Eosin (H&E) or 5% cresyl violet (Nissl). H&E sections are used to evaluate lesion volume, while Nissl is used to assess cell loss due to injury. In each case, the contralateral side serves as a control for the injured side of the brain.

For contusion volume analysis, a single section taken every millimeter from 1.3 mm to 6.3 mm posterior to Bregma is examined under low magnification. Image analysis software (e.g. MCID/M4 image software, or NIH image) may be used to capture the images, and to calculate hemispheric area of the ipsilateral and contralateral side of each section. The volume of the ipsilateral and contralateral hemispheres is then computed by integrating the area of each section and the distance between sections.

To assess cell loss following injury, cell counts are performed in the CA3 hippocampal region. Nissl stained sections are examined at moderate magnification, and cells with neuronal morphology in the CA3 region along an arc of defined length are counted. The number of cells obtained is compared to the number of neurons counted along a similar arc in CA3 of the contralateral (uninjured) side to determine the degree of cell loss on the injured side.

Data Analysis

For analysis of continuous variables compared across groups, (e.g. watermaze latency) data is examined using analysis of variance (ANOVA) followed by post-hoc Newman-Keuls tests. Ordinal measurements such as combined neuroscores are analyzed using the non-parametric Kruskal-Wallis ANOVA followed by post-hoc non-parametric Mann-Whitney U-tests.

Results:

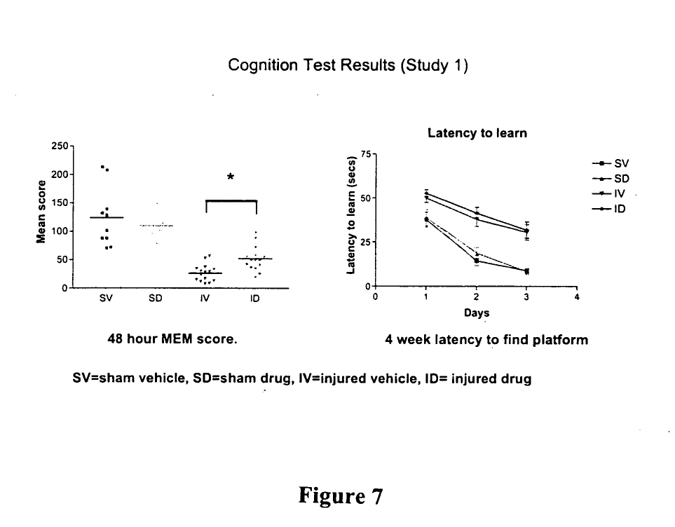

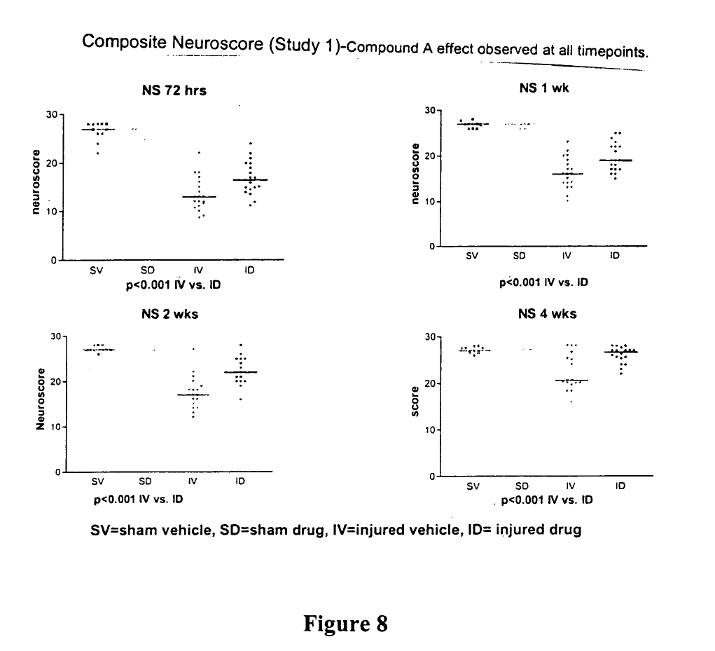

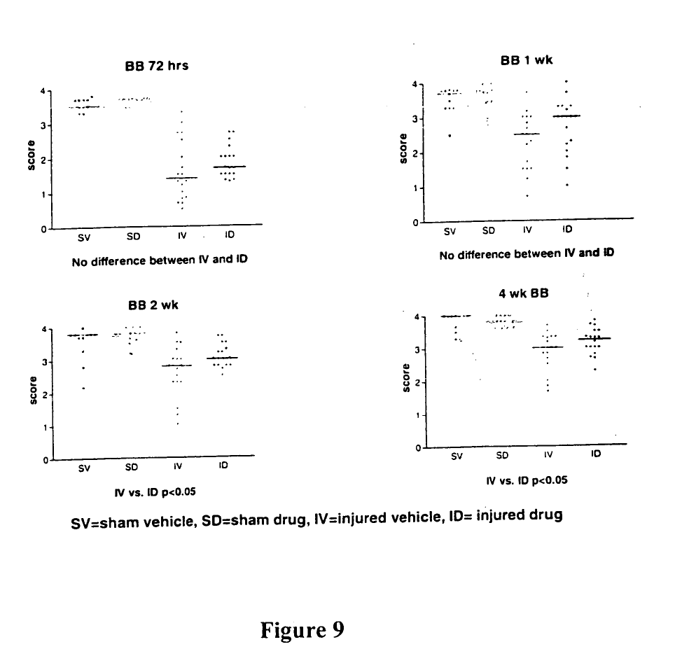

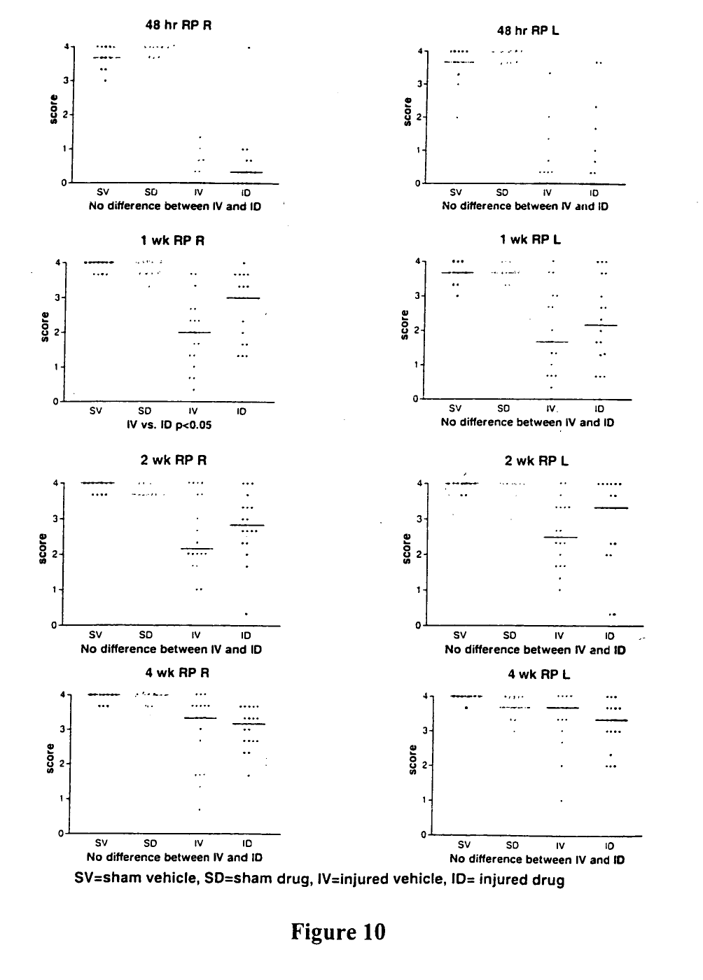

Compound A was tested twice in the fluid percussion injury model as described above. In one instance, Compound A was formulated in 75% PEG 200 and administered intraperitoneally twice daily for 7 days starting 15 minutes post-injury at a dose of 20 mg/kg/BID. Compound A treatment improved memory function at 48 hours post-injury although it had no effect on a learning paradigm tested at 4 weeks post-injury. The compound had robust acute and long term effects on the composite neuroscore, but effects on the other locomotor tests, beam balance and rotating pole, were limited. Compound A treatment did not attenuate oedema formation at 48 hours. (See FIGS. 7 through 10 and Table 2 for results of study number 1)

TABLE 2
Summary of Study Number 1

| | 48 hrs | 72 hrs | 1 wk | 2 wk | 3 wk | 4 wk |
|---|---|---|---|---|---|---|
| MEM | + | | | | | |
| Oedema | − | | | | | |
| NS | − | + | + | + | + | + |
| BB | − | − | − | + | − | + |
| RP | − | − | +/− | − | − | − |
| Latency | | | | | | − |

MEM: Morris Watermaze Memory Paradigm
NS: composite Neuroscore
BB: Beam Balance
RP: Rotating Pole
Latency: Morris Water Maze Learning Paradigm

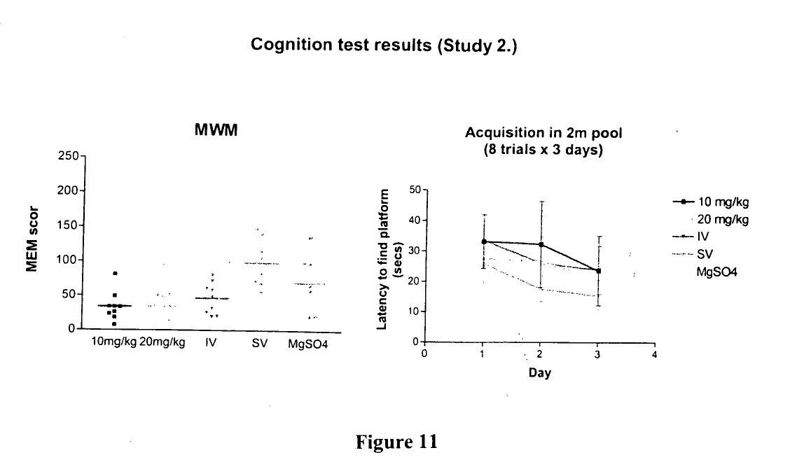

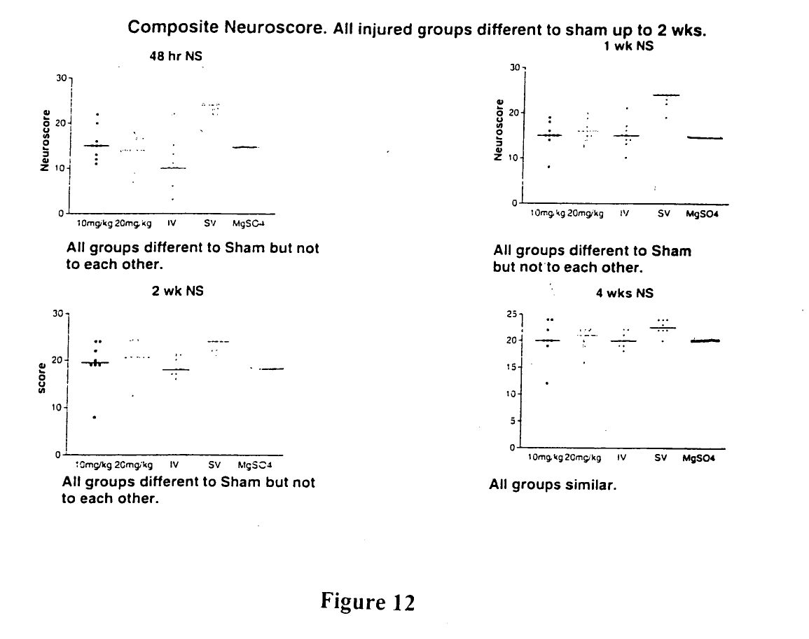

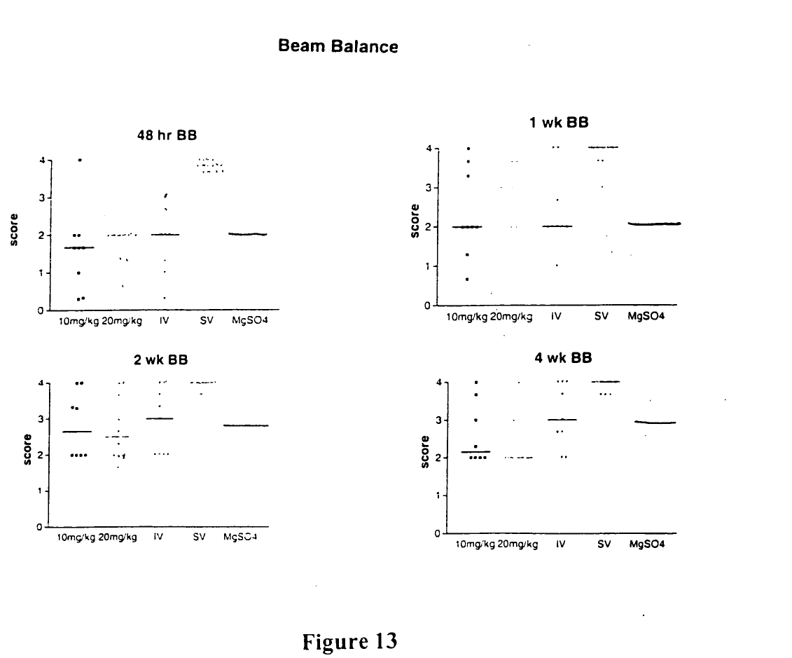

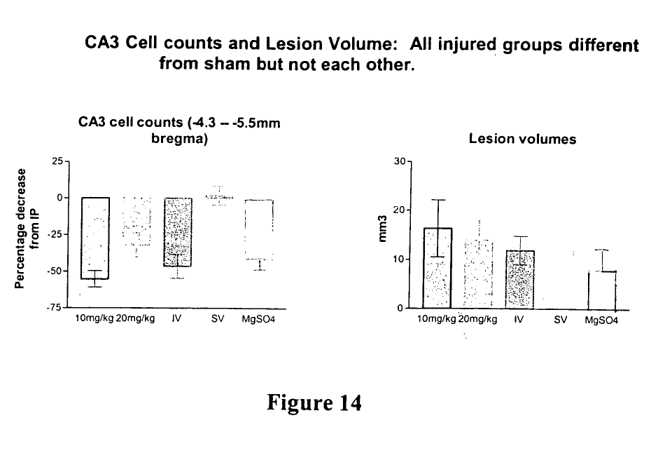

In the second test, Compound A was formulated in 50% PEG 400 and 25% ethanol and was administered intraperitoneally twice daily for 5 days starting at 15 minutes post injury at 10 mg/kg/BID and 20 mg/kg/BID. No effects of the compound were observed in acute or long term locomotor or cognitive tests. Further, there was no attenuation of CA3 cell loss or lesion volume in Compound A treated animals compared to controls. (See FIGS. 11 through 14 and Table 3 for results)

TABLE 3
Summary of Study Number 2

| | 48 hrs | 72 hrs | 1 wk | 2 wk | 3 wk | 4 wk |
|---|---|---|---|---|---|---|
| MEM | − | | | | | |
| Oedema | − | | | | | |
| NS | − | − | − | − | − | − |
| BB | − | − | − | − | − | − |
| Latency | | | | | | − |
| Lesion vol | | | | | | − |
| CA3 cell | | | | | | − |
| loss | | | | | | |

MEM: Morris Watermaze Memory Paradigm
NS: composite Neuroscore
BB: Beam Balance
RP: Rotating Pole
Latency: Morris Water Maze Learning Paradigm

The differences in the results obtained from the two TBI studies may be due to differences in formulation of the compound or in the difference in the anesthetic used during surgery eg. the first test used pentobarbital, whereas the second test used isofluorane. It is possible that the different formulations may result in differences in bioavailability of the drug, thus leading to the different outcomes in the two tests. Alternatively, it may be possible that certain anesthetics exert a neuroprotective effect on their own. Both pentobarbital and isoflurane have been reported to have some neuroprotective activity, which could influence the therapeutic range of the drug and therefore limit the comparability of the two studies.

### EXAMPLE 23

Spinal Cord Injury Model

Compound A was tested in both the rat dorsal hemisection (Guth, L. et al. (1980), J. Neurosurgery 52: 73-86) and contusion models (Scheff, S. W. (2003), J. Neurotrauma 20: 179-193) for evaluation of efficacy. These models are widely used in the field of spinal cord injury and replicate many of the pathological aspects found in humans who have sustained a spinal cord injury. Rats were subjected to an experimental spinal cord injury at T8, and were given Compound A shortly thereafter. Behavioral tests to assess the locomotion deficits were performed after the lesion. At the end of the experiment, animals were sacrificed and the spinal cord was collected for histopathological analysis. A comparison was then made between Compound A-treated and untreated animals to determine if administration of the compound resulted in an improvement in behavioral functions. Histological assessment of the tissue at the site of injury was also made in both the drug treated and untreated group. In particular, subsequent to the behavioral measurement, spinal cord tissue from each animal was obtained and serial transverse tissue sections were stained with either anti-neurofilament antibody or antibodies specific for the inflammatory markers, tumor necrosis factor alpha (TNFα) and for the inducible form of nitric oxide synthetase (iNOS), followed by incubation with Alexa Fluor®-conjugated secondary antibodies.

Experimental Protocol

Female rats (Sprague-Dawley, 250-300 g) were purchased from Charles River. One week before spinal cord injury, the animals were both trained and had baseline measures taken on a locomotor behavioral test, the BBB test (Beattie Basso Bresnahan method) (Basso, D. M. et al. (1995), J. Neurotrauma 12(1): 1-21).

The BBB score is a 21-point scale representing 21 stages of locomotor recovery after spinal cord injury. The scale is based on unique combinations of scored behaviors, ranked according to time of appearance after injury. The score can be divided into three parts: from 0-8, the scores emphasize voluntary movements of hindlimb joints; from 8-14, the scores represent standing and stepping with progressively better forelimb-hindlimb coordination; from 15-21, the scores indicate greater strength and better foot placement and balance. Each score represented a unique combination of behaviors, providing a non-ambiguous ordinal scale. The scale is described in Table 4.

Every week, the rats were placed in a standard open field (a plastic tub with walls) and observed by trained investigators from two sides for several minutes. Characteristics of locomotion were checked off on a scoring sheet and the final score represents the consensus opinion of the observers. Detailed inter-rater reliability analyses indicate that experienced observers can achieve a standard deviation of less than 1 point on the scale. All scoring was done blinded, that is, by people who were not aware of the treatments received by individual rats. Treatments were masked through the analysis.

TABLE 4
Summary of BBB scoring method

| BBB scores | | Comments |
|---|---|---|
| 0 | No observable hindlimb (HL) movement | None |
| 1 | Slight movement on one or two HL joints | Slight 50% of joint range |
| 2 | Extensive movement of one HL joint and | Extensive 50% of joint range |
| | slight movement of the other joint | |
| 3 | Extensive movement of two HL joints | Two joints = usually hip & knee |
| 4 | Slight movement of three HL joints | Three joints = hip, knee & ankle |
| 5 | Slight movement of two HL joints & | |
| | extensive movement of third HL joint | |
| 6 | Extensive movement of two joints HL | Third joint = usually the ankle |
| | joints & slight movement of third HL joint | |
| 7 | Extensive movement of all three HL joints | |
| 8 | Sweeping with no weight support or | Sweeping = rhythmic 3 joint |
| | Plantar placement with no weight support | movement |
| 9 | Plantar placement with weight support OR | Weight support = HL extensor |
| | Dorsal stepping with weight support | contraction with elevation of |
| | | hindquarters in stance |
| 10 | Occasional weight supported steps with no | Occasional = >5% & 50% |
| | forelimb-hindlimb (FL-HL) coordination | Steps = plantar steps with weight |
| | | support |
| 11 | Frequent to consistent steps (FCS) with no | Frequent = 51-94% of the time |
| | coordination | Consistent = 95-100% of the |
| | | time |
| 12 | FCS with occasional coordination | 6-50% bouts of locomotion |
| | | coordinated |
| 13 | FCS with frequent coordination | 51-95% bouts of locomotion |
| | | coordinated |
| 14 | Consistent coordinated steps (CCS) & | Rotated = internal or external |
| | paw rotated on placement & liftoff OR | rotation |
| | Frequent steps, consistent coordination | |
| | With occasional dorsal steps | |
| 15 | CCS & no or occasional toe clearance & | Parallel = paw placement to body |
| | parallel paw position on initial placement | Toe clearance = steps without toe |
| | | drag |
| 16 | CCS & frequent toe clearance | Frequent toe clearance 50% no |
| | | toe drag |
| 17 | CCS & parallel paw on placement and | |
| | liftoff | |
| 18 | CCS & consistent toe clearance | Consistent toe clearance 4 toe |
| | | drags |
| 19 | CCS & parallel paw on placement and | Tail down = touches ground |
| | liftoff | when walking |
| | Tail down part or all the time | |
| 20 | CCS & parallel paw on placement and | Trunk instability = lateral weight |
| | liftoff | shifts, waddling, lurching |
| | Tail consistently up, trunk unstable | |
| 21 | CCS, consistent toe clearance, parallel | Consistent trunk stability no |
| | paws, tail consistent up, consistent trunk | lurching |
| | stability | |

Spinal Cord Injury Procedure

On the day of surgery, animals were placed upon a heated surgical surface, and a laminectomy of the T7 vertebrae was performed. Once exposed, the spinal cord (T8, corresponds to T7 vertebrae) was subjected to an injury consisting of either a dorsal hemisection or contusion below. The dorsal spinal cord injury studies are summarized below.

14 Day Dorsal Hemisection Study (SCI Study No. 1)

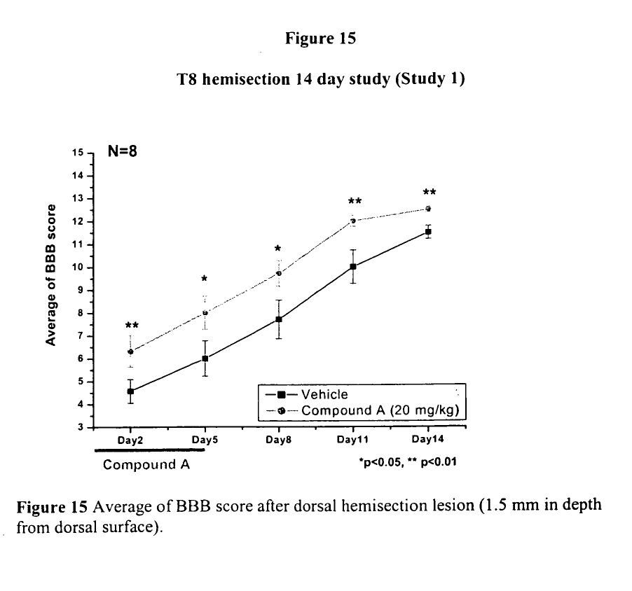

In one study (SCI Study No. 1), the animals received a dorsal hemisection lesion, which consisted of a lesion of 1.5 mm from the dorsal surface. This was a 14 day study. The results of this study, which can be found in FIG. 15, demonstrated that administration of Compound A resulted in significant improvement in neurological function, as shown by an increase in the BBB score.

21 Day Over Dorsal Hemisection Study (SCI Study No. 2)

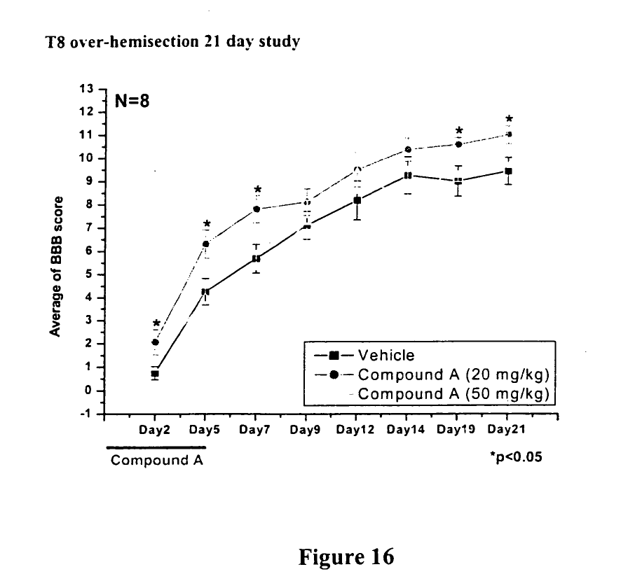

A second study was conducted for 21 days (SCI Study No. 2) using an over dorsal hemisection. In this study, the animals received a lesion, which was 2 mm in depth from the dorsal surface. This represents a more severe injury than the regular dorsal hemisection model, in which the lesion is only 1.5 mm from the dorsal surface. SCI study Nos. 1 and 2 used the first formulation of Compound A (see below). The results of this study, which can be found in FIG. 16, demonstrated that administration of Compound A at a dose of 20 mg/kg BID, resulted in significant improvement in neurological function, as shown by an increase in the BBB score.

5 Week Over Dorsal Hemisection Study (SCI Study No. 3)

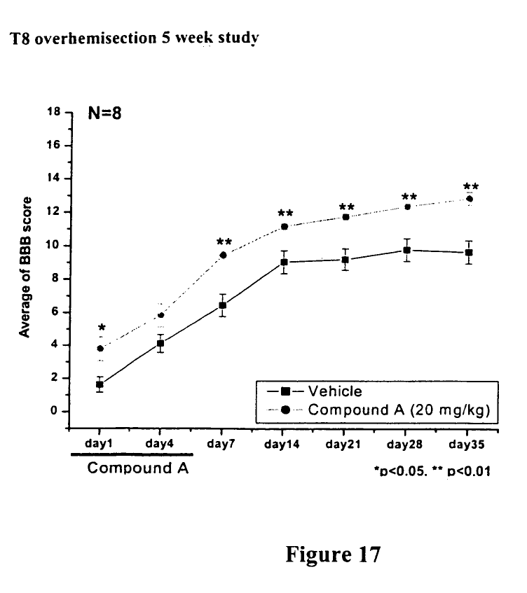

A third study (SCI Study No. 3) was conducted, which was a 5 week study using the second formulation of Compound A (see below). The results of this study, which can be found in FIG. 17, also demonstrated that administration of Compound A at a dose of 20 mg/kg BID, resulted in significant improvement in neurological function, as shown by an increase in the BBB score.

14 Day Contusion Study (SCI Study No. 4)

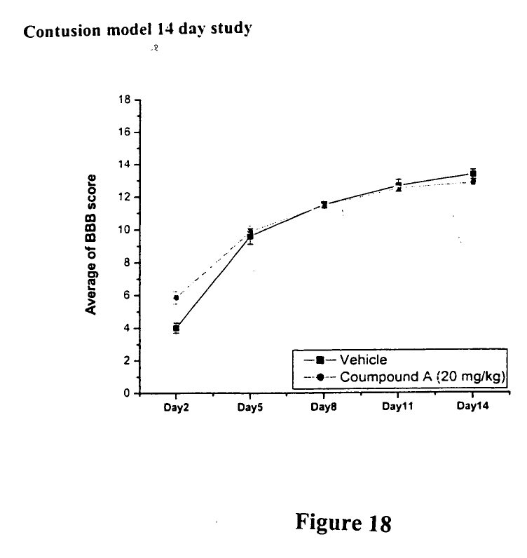

The fourth study was conducted in rats having received a contusion to the spinal cord followed by administration of either vehicle or Compound A at a dose of 20 mg/kg BID. Assessment of functional neurological outcome was made using the BBB score over 14 days. The results shown in FIG. 18 demonstrate that no significant effects were observed in this particular study. The reasons for this are unclear at this time.

Procedure for Detection of TNF-α and iNOS

Histological assessment of the tissue at the site of injury was also made in both the drug treated and untreated group. In particular, subsequent to the behavioral measurement, spinal cord tissue from each animal was obtained and serial transverse tissue sections were stained with either goat anti-rat TNF-α antibody diluted at 1:25 or 1:50 (R& D system), or mouse anti-rat iNOS antibody at 1:500 dilution, followed by staining with either Alexa Fluor®-conjugated donkey anti-goat IgG (for the TNF-α assessment), or Alexa Fluor®-conjugated goat anti-mouse IgG (for iNOS assessment). The procedure is as follows: Slides are baked at 55° C. for 25 min, washed in permeabilization buffer (1×TBS in 0.5% Triton) and blocked for nonspecific binding with 5% normal donkey serum, 1% BSA, 0.5% Triton in 1×PBS. Slides are incubated with 1^{st} antibody overnight at 4° C., washed in TBS, 3×10 min and then incubated with 2^{nd} Ab for 2 hr at Room Temperature. Sections are washed in TBS, 3×10 min, soaked in ddH₂O and coverslipped with Fluoromount G.

Preparation of Compound A and Dosing

Two preparations of Compound A were made and compared. The first formulation was prepared by dissolving the compound in 75% PEG200 and 25% dH₂O, followed by sonication in a water bath to make a suspension of the compound. This formulation was used for SCI study Nos. 1, 2 and 4. The second formulation was prepared by first dissolving the compound in ethanol, and then diluting with PEG400 and dH₂O to a final formulation of 50% PEG400, 25% ETOH, and 25% dH₂O. This formulation was used in SCI Study No. 3. Compound A was administered intraperitoneally 1 hr after the surgery, and dosing was repeated twice a day (BID) for 5 days at dose levels of 20 mg/kg/BID (total daily dose: 40 mg/kg/day) or 50 mg/kg/BID (total daily dose: 100 mg/kg/day).

Results

Compound A and Assessment of its Effects on Locomotor Function

Locomotor function was assessed by means of the BBB score, an ordinal scale from 1-21 at intervals of 3-7 days for up to 3 weeks. BBB behavioral testing started from day 1 following the surgery and continued weekly up to 5 weeks post-surgery. The results showed that Compound A improved locomotor function in animals receiving 20 mg/kg/BID (40 mg/kg/day) formulated in 75% PEG 200 administered intraperitoneally twice a day for 5 days.

This compound demonstrated robust acute and long-term effects up to 5 weeks on the BBB locomotor test in the over dorsal hemisection model (FIG. 17). This particular experiment was performed by administration of 20 mg/kg of Compound A in a formulation consisting of 50% PEG400, 25% ETOH, and 25% dH₂O. Dosing was given interperitoneally 1 hr after the surgery, and repeated for 5 days, twice a day.

As shown herein, Compound A, in two different formulations, demonstrated significant improvement in neurological function in the hemisection model as shown by an increase in the BBB score.

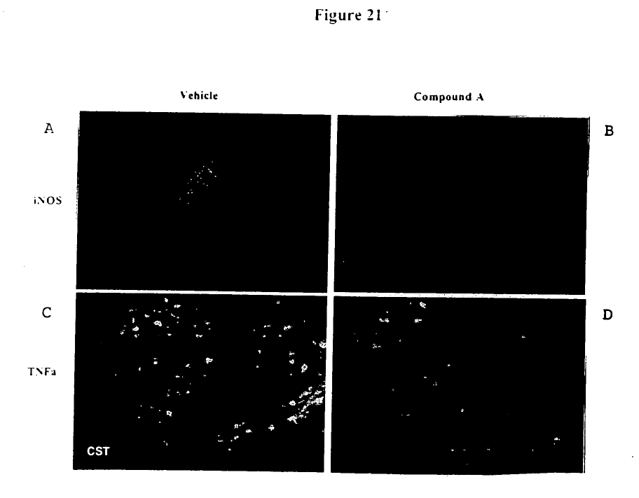

Significant differences in locomotor function between the drug treated and the vehicle control groups were not observed in the contusion model for SCI (FIG. 18) while inflammatory markers were reduced in drug treated animals (FIG. 21). Although the reasons for the differences observed with respect to drug efficacy on locomotor function between the two SCI models are unknown at this time, one may speculate that the difference in results may be due to the different nature of the injury in the contusion injury model compared to the hemisection model.

Results of Histopathological Analysis

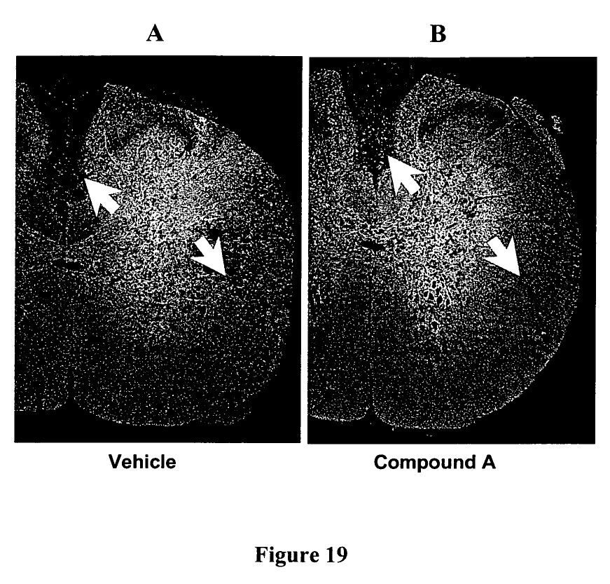

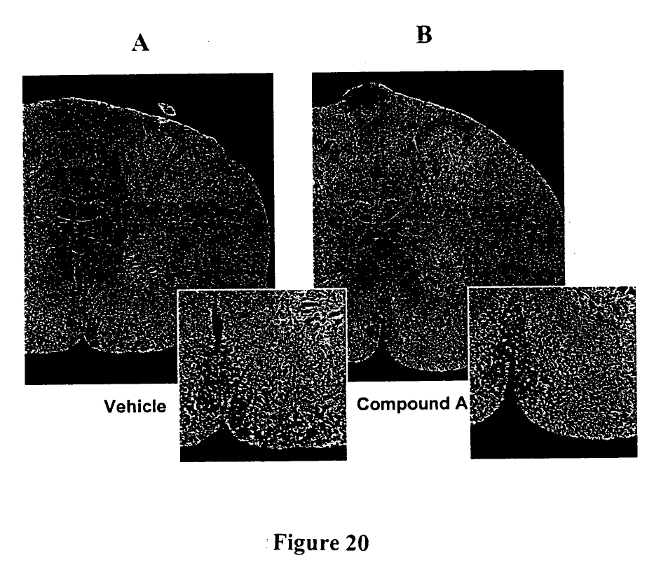

As shown in FIGS. 19A and B, and FIGS. 20A and B, spinal cord tissue obtained from Compound A treated animals (B) showed significantly higher nerve fiber density as compared to the vehicle control (untreated) animals. In particular, FIGS. 19A and B shows marked differences between the drug treated and control animals particularly in the lateral white matter rostral to the epicenter. Furthermore, there were also marked differences between the drug treated and the control animals in the ventral white matter in sections caudal to the epicenter (FIGS. 20A and B).

As shown in FIG. 21, inflammatory markers TNFα and iNOS were decreased in the region of the corticospinal tract in Compound A treated animals (Panels D and B, respectively) as compared to the untreated control group (Panels C and A, respectively) after contusion injury.

Conclusions in the Spinal Cord Injury Model

The results as shown herein support the beneficial effect of Compound A in neurological recovery in an experimental spinal cord injury model. These results are acute and long lasting. Decreased inflammatory markers are found associated with this recovery.

### EXAMPLE 24

### Blood Brain Disposition of Compound A in the Mouse, Rat and Squirrel Monkey

Blood-brain distribution of Compound A was assessed in the mouse, rat, and squirrel monkey by determining the concentrations of Compound A in blood and brain tissue at specific times post dose. Rats and mice received a single po dose of 30 mg/kg. The monkeys received a twice-daily 10 mg/kg po dose of Compound A for a total of 5 weeks prior to sacrifice. Brain to blood ratios for Compound A, as shown below in Table 4, show high penetration of the blood-brain barrier.

TABLE 5
Brain-Blood Distribution of Compound A in the Rat, Mouse and
Monkey

| | Time | Brain/Blood Ratio |
|---|---|---|
| Species | Post-Dose (h) | (Mean ± SD) |
| Rat | 0.5 | 0.71 ± 0.03 |
| Rat | 1.0 | 0.66 ± 0.02 |
| Rat | 2.0 | 0.67 ± 0.03 |
| Mouse | 0.5 | 0.79 ± 0.05 |
| Mouse | 1.0 | 0.80 ± 0.05 |
| Mouse | 2.0 | 0.76 ± 0.06 |
| Monkey^{a} | 24 | 0.88 ± 0.09 |

Note:
Number of animals per time point: rats and mice, n = 5; monkeys, n = 12. Brains were not perfused prior to removal of tissue samples.
^{a}Monkeys also received a single sc injection of saline, or 1.0 or 2.0 mg/kg MPTP on Day 7.

Results:

From the results in Table 5, it is evident that Compound A, and the compounds of the invention, demonstrate the ability to traverse the blood brain barrier in a substantial manner and amount. Such ability is a significant indicator and characteristic of, among other things, agents that can rapidly act to provide effective treatment to injuries and dysfunctions either involving or localized to the brain, such as TBI, and thereby further demonstrates the therapeutic activity of the compounds of the invention.

From the foregoing description, various modifications and changes in the compositions and methods of this invention will occur to those skilled in the art. All such modifications coming within the scope of the appended claims are intended to be included therein.

What is claimed is:

1. A pharmaceutical composition for the treatment of pain comprising an effective pain-treating amount of a benzamide compound in a pharmaceutically acceptable carrier,
wherein the benzamide is a material of Formula I: 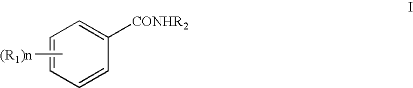
where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5.

2. A pharmaceutical composition for the treatment of traumatic injury comprising an effective traumatic injury-treating amount of a benzamide compound in a pharmaceutically acceptable carrier,
wherein the benzamide is a material of Formula I: 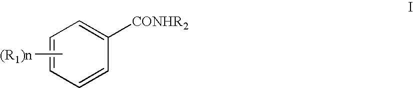
where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5.

3. The composition of either of claims 1 or 2 wherein the benzamide is selected from the group consisting of acetamidobenzamides, aminobenzamides and nitrobenzamides.

4. The composition of claim 3 wherein the benzamide is selected from the group consisting of:
N-tert-butyl-4-acetamidobenzamide (Compound A);
N-iso-propyl-4-acetamidobenzamide (Compound B);
N-tert-amyl-4-acetamidobenzamide (Compound C);
N-cyclopropylmethyl-4-acetamidobenzamide (Compound E);
N-iso-propyl-4-nitrobenzamide (Compound F);
N-tert-butyl-3-nitrobenzamide (Compound G);
N-tert-butyl-2-nitrobenzamide (Compound H);
N-n-butyl-4-nitrobenzamide (Compound J);
N-n-propyl-4-nitrobenzamide (Compound K);
N-tert-butyl-3,5-dinitrobenzamide (Compound L);
N-1-methylpropyl-4-nitrobenzamide (Compound M);
N-tert-butyl-4-aminobenzamide (Compound N);
N-tert-butyl-3-aminobenzamide (Compound P); and
N-tert-butyl 4-nitrobenzamide (Compound Q).

5. The composition of claim 4 wherein the benzamide is N-tert-butyl-4-acetamidobenzamide (Compound A).

6. The composition of claim 2 wherein the traumatic injury is selected from the group consisting of traumatic brain injury and acute spinal cord injury.

7. A method for treating pain in a mammalian subject in need thereof, comprising delivering/administering to said subject an effective pain-treating amount of a pharmaceutical composition comprising a benzamide.

8. The method of claim 7, wherein said benzamide has a formula as set forth in I: 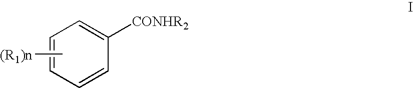
where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5 atoms in a pharmaceutically acceptable carrier.

9. The method of claim 7, wherein said pain is acute pain.

10. The method of claim 9, wherein said pain includes post-operative pain, shock, pain resulting from inflammation, pain resulting from trauma, acute breakthrough pain associated with a chronic pain condition, and combinations thereof.

11. The method of claim 7, wherein said pain is chronic pain.

12. The method of claim 11, wherein said pain includes headache pain, back pain, sciatica, cancer pain, arthritis pain, neuropathic pain, psychogenic pain, and combinations thereof.

13. The method of claim 7, wherein said pharmaceutical composition is delivered/administered via a route selected from the group consisting of topical, oral, caudal, subcutaneous, intramuscular, intravenous, perineural, intraperitoneal, epidural intranasal, intracranial, local intracerebral, intrathecal, intraventricular, transdermal, and combinations thereof.

14. The method of claim 7, wherein said pharmaceutical composition is delivered/administered to the central nervous system of said subject.

15. The method of claim 14, wherein delivery/administration to the central nervous system is selected from the group consisting of intracranial, local intracerebral, intrathecal, and intraventricular delivery/administration.

16. The method of claim 7, wherein the pharmaceutical composition delivers an effective amount directly to the central nervous system without adversely affecting other tissues or organ systems.

17. The method of claim 13, wherein the pharmaceutical composition is administered by an infusion pump.

18. The method of claim 13, wherein the pharmaceutical composition is administered in an aerosol formulation.

19. The method of claim 13, wherein the pharmaceutical composition is administered over a period of at least several days.

20. The method of claim 13, wherein the pharmaceutical composition is administered over a period of at least four weeks.

21. The method of claim 13, wherein the pharmaceutical composition is administered over a period of at least three months.

22. The method of claim 13, wherein the pharmaceutical composition is administered on an as needed basis.

23. The method of claim 13, wherein the pharmaceutical composition is administered as a sustained release formulation or in a formulation that is capable of crossing the blood brain barrier.

24. The method of claim 7, wherein the pain is associated with a condition selected from the group consisting of diabetic neuropathy, herpes zoster, fibromyalgia AIDS, syphilis, neuritis, temporomandibular disorder, back pain, myofacial pain, acute spinal cord injury, traumatic brain injury, cancer pain, visceral pain, intraneural inflammation, optic neuropathy, neuropathy secondary to nerve trauma, post herpetic neuralgia, reflex sympathetic dystrophy, and ankylosing spondylitis.

25. A method for producing local anesthesia or analgesia in a mammal, the method comprising administering to a mammal the pharmaceutical composition of claim 7.

26. The method of claim 25, wherein the local anesthesia or analgesia is effective for treatment of conditions associated with increased sensory functions, said conditions selected from the group consisting of hyperesthesia, hyperalgesia, dyesthesia, allodynia, tinnitus and ganglionic dysfunction.

27. The method of claim 26, wherein the local anesthesia or analgesia is administered topically to a skin region having or manifesting neuropathic pain.

28. The method of claim 27, wherein said treatment of neuropathic pain in a skin region of a mammal comprises the application to said skin region a transdermal patch containing said pharmaceutical composition.

29. A method for treating a patient suffering from a traumatic injury comprising administering to said patient an effective amount of a pharmaceutical composition comprising a benzamide compound of the formula I: 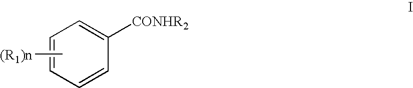
where n is 1 or 2, R₁ is a group selected from acetyl, alkyl, amino, acylamino (NHCOR₃), halo, nitro, and trifluoroalkyl, R₂ is saturated alkyl of 3 to 5 atoms, and R₃ is alkyl of 1 to 5 atoms in a pharmaceutically acceptable carrier.

30. The method of claim 29, wherein said traumatic injury is an injury to the central nervous system.

31. The method of claims 8 or 29 wherein R₁ is amino, acylamino (NHCOR₃), or nitro.

32. The method of claim 31 wherein R₂ is t-Bu.

33. The method of claim 32 wherein n is 1.

34. The method of claims 8 or 29 wherein n is 1, R₁ is 4-amino, 4-acylamino (NHCOR₃), or 4-nitro and R₂ is t-Bu.

35. The method of claims 8 or 29 wherein said composition is administered orally.

36. The method of claims 8 or 29 wherein said composition is administered parenterally.

37. The method of claim 36, wherein said parenteral administration is selected from the group consisting of topical, caudal, subcutaneous, intramuscular, intravenous, perineural, intraperitoneal, epidural, intranasal, intracranial, local intracerebral, intrathecal, intraventricular, transdermal, and combinations thereof.

38. The method of claim 37, wherein said administration is by infusion pump.

39. The method of claims 8 or 29 wherein said composition is administered rectally.

40. The method of claim 29, wherein said traumatic injury is selected from the group consisting of traumatic brain injury and spinal cord injury.

41. The method of claim 40, wherein the spinal cord injury is an acute or chronic spinal cord injury.

42. The method of claims 8 or 29, wherein said benzamide is selected from the group consisting of:
N-tert-butyl-4-acetamidobenzamide (Compound A);
N-iso-propyl-4-acetamidobenzamide (Compound B);
N-tert-amyl-4-acetamidobenzamide (Compound C);
N-methylcyclopropyl-4-acetamidobenzamide (Compound E);
N-iso-propyl-4-nitrobenzamide (Compound F);
N-tert-butyl-3-nitrobenzamide (Compound G);
N-tert-butyl-2-nitrobenzamide (Compound H);
N-n-butyl-4-nitrobenzamide (Compound J);
N-n-propyl-4-nitrobenzamide (Compound K);
N-tert-butyl-3,5-dinitrobenzamide (Compound L);
N−1-methylpropyl-4-nitrobenzamide (Compound M);
N-tert-butyl-4-aminobenzamide (Compound N);
N-tert-butyl-3-aminobenzamide (Compound P); and
N-tert-butyl 4-nitrobenzamide (Compound Q).

43. The method of claim 42 wherein said benzamide is N-tert-butyl-4-acetamidobenzamide (Compound A).

44. A method for treating or preventing pain in a mammal comprising administering to said mammal an effective pain treating or preventing dose of a pharmaceutical composition according to any of claims 1 and 3-5.

45. A method for the prophylactic treatment of patients susceptible to outbreaks of shingles or for patients scheduled to receive chemotherapy comprising administering a therapeutically effective amount of the composition of claim 1.

46. A method for the treatment of traumatic injury to the brain or spinal cord of a mammal comprising administering to said mammal an effective brain or spinal cord injury treating dose of a pharmaceutical composition according to any of claims 2-5.

47. The method of claims 44 or 46, wherein the mammal is a human.
